SEQUENCE LISTING (9 sequences)

SEQ ID NO: 1 - protein, 170 aa, Eimeria acervulina
MGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 2 - DNA, 635 bp, synthetic construct
  source 1..635: mol_type = other DNA
accatggcgaacaagcacctgagccttagcctcttcctcgtgctcctgggcctctccgccagcctcgcctccggcggggaggaggcggacacccaggcctgggacacctcggtgaaggagtggctcgtcgacaccggcaaggtgtacgctggcggcatcgccagcatcgccgacggctgcaggctgttcggcgctgccatcgacaacggcgaggacgcctggtcccagctcgtcaagacgggctaccagatcgaggtgctgcaggaggacggcagctccacgcaggaggactgcgacgaggctgagacgctgaggcaggccatcgtggacggcagggccccgaacggcgtgtacatcggcggcatcaagtacaagcttgcggaggtcaagagggacttcacctacaacgaccagaactacgacgtggccatcctcggcaagaacaagggcggcggcttcctcatcaagacgcccaacgacaacgtggtcatcgcgctctacgacgaggagaaggagcagaacaaggccgacgctctcacgactgctctcgctttcgcggagtacctctaccagggcggcttctgataggttaacctagacttgtccatcttctggattggccaacttaattaatt

SEQ ID NO: 3 - DNA, 647 bp, synthetic construct
  source 1..647: mol_type = other DNA
accatggcgaacaagcacctgagccttagcctcttcctcgtgctcctgggcctctccgccagcctcgcctccggcggggaggaggcggacacccaggcctgggacacctcggtgaaggagtggctcgtcgacaccggcaaggtgtacgctggcggcatcgccagcatcgccgacggctgcaggctgttcggcgctgccatcgacaacggcgaggacgcctggtcccagctcgtcaagacgggctaccagatcgaggtgctgcaggaggacggcagctccacgcaggaggactgcgacgaggctgagacgctgaggcaggccatcgtggacggcagggccccgaacggcgtgtacatcggcggcatcaagtacaagcttgcggaggtcaagagggacttcacctacaacgaccagaactacgacgtggccatcctcggcaagaacaagggcggcggcttcctcatcaagacgcccaacgacaacgtggtcatcgcgctctacgacgaggagaaggagcagaacaaggccgacgctctcacgactgctctcgctttcgcggagtacctctaccagggcggcttcaaggacgagctgtgataggttaacctagacttgtccatcttctggattggccaacttaattaatt

SEQ ID NO: 4 - protein, 450 aa, Eimeria tenella
MGKEKTHINLVVIGHVDSGKSTTTGHLIYKLGGIDKRTIEKFEKESSEMGKASFKYAWVLDKLKAERERGITIDIALWQFETPAFHYTVIDAPGHRDFIKNMITGTSQADVALLVVPADQGGFEGAFSKEGQTREHALLAFTLGVKQMIVGINKMDATSPEKYSEARFNEIQAEVSRYLKTVGYNPEKVPFVPISGFVGDNMVERSSNMGWYKGKTLVEALDSVEPPKRPVDKPLRLPLQDVYKIGGIGTVPVGRVETGVLKPGMVVTFAPSGLQTEVKSVEMHHAQLEQAVPGDNVGFNVKNVSVKDVKRGHVASDSKNDPAKAAASFQAQVIVLHHPGQINPGYTPVLDCHTAHISCKFADLEKRLDRRSGKALEDSPKSIKSGDAAIVRMEPSKPMCVEAFIEYPPLGRFAVRDMKQTIAVGVIKAVEKKEAGGKVTKSAQKAAAKK

SEQ ID NO: 5 - DNA, 1475 bp, synthetic construct
  source 1..1475: mol_type = other DNA
accatggcgaacaagcacctgagccttagcctcttcctcgtgctcctgggcctctccgccagcctcgcctccggcggcaaggagaagacgcacatcaacctggtggtcatcggccacgtggactccggcaagagcaccacgaccggccacctgatctacaagctcggcggcatcgacaagcgcaccatcgagaagttcgagaaggagtccagcgagatgggcaaggcgagcttcaagtacgcctgggtcctggacaagctcaaggccgagagggagaggggcatcacgatcgacatcgcgctctggcagttcgagacgcccgccttccactacaccgtgatcgacgcccccggccacagggacttcatcaagaacatgatcaccggcaccagccaggcggacgtcgccctcctggtggtgcccgcggaccagggcgggttcgagggcgcgttctccaaggaggggcagacgagggagcacgccctcctggccttcaccctgggcgtgaagcagatgatcgtcggcatcaacaagatggacgccacgagccccgagaagtactccgaggcgcgcttcaacgagatccaggccgaggtgtcccgctacctcaagaccgtcggctacaacccggagaaggtgccgttcgtccccatctccggcttcgtgggcgacaacatggtcgagcgctccagcaacatgggctggtacaagggcaagaccctggtggaggccctggactcggtggagccgccaaagcgcccggtggacaagccactcaggctgccgctccaagacgtctacaagatcggtggcatcggcaccgtgccggtgggcagggtggagacgggcgtcctgaagccgggcatggtggtcaccttcgccccctccggcctccagacggaggtgaagtccgtcgagatgcaccacgcccagctggagcaggcggtgcccggcgacaacgtgggcttcaacgtcaagaacgtgtccgtcaaggacgtgaagaggggccacgtcgcctccgacagcaagaacgacccggccaaggcggccgccagctttcaggcccaggtcatcgtgctccaccaccccggccagatcaacccgggctacacccccgtgctggactgccacacggcccacatcagctgcaagttcgcggacctggagaagaggctggacaggaggtcgggcaaggctctggaggactccccgaagagcatcaagtcgggcgacgccgctatcgtgaggatggagccgagcaagcccatgtgcgtcgaggcgttcatcgagtacccgcccctcggcaggttcgccgtgagggacatgaagcagaccatcgcggtgggcgtcatcaaggccgtcgagaagaaggaggcgggcggcaaggtgacgaagtcggctcagaaggccgcggctaagaagtgataggttaacctagacttgtccatcttctggattggccaacttaattaata

SEQ ID NO: 6 - DNA, 1484 bp, synthetic construct
  source 1..1484: mol_type = other DNA
accatggcgaacaagcacctgagccttagcctcttcctcgtgctcctgggcctctccgccagcctcgcctccggcggcaaggagaagacgcacatcaacctggtggtcatcggccacgtggactccggcaagagcaccacgaccggccacctgatctacaagctcggcggcatcgacaagcgcaccatcgagaagttcgagaaggagtccagcgagatgggcaaggcgagcttcaagtacgcctgggtcctggacaagctcaaggccgagagggagaggggcatcacgatcgacatcgcgctctggcagttcgagacgcccgccttccactacaccgtgatcgacgcccccggccacagggacttcatcaagaacatgatcaccggcaccagccaggcggacgtcgccctcctggtggtgcccgcggaccagggcgggttcgagggcgcgttctccaaggaggggcagacgagggagcacgccctcctggccttcaccctgggcgtgaagcagatgatcgtcggcatcaacaagatggacgccacgagccccgagaagtactccgaggcgcgcttcaacgagatccaggccgaggtgtcccgctacctcaagaccgtcggctacaacccggagaaggtgccgttcgtccccatctccggcttcgtgggcgacaacatggtcgagcgctccagcaacatgggctggtacaagggcaagaccctggtggaggccctggactcggtggagccgccaaagcgcccggtggacaagccactcaggctgccgctccaagacgtctacaagatcggtggcatcggcaccgtgccggtgggcagggtggagacgggcgtcctgaagccgggcatggtggtcaccttcgccccctccggcctccagacggaggtgaagtccgtcgagatgcaccacgcccagctggagcaggcggtgcccggcgacaacgtgggcttcaacgtcaagaacgtgtccgtcaaggacgtgaagaggggccacgtcgcctccgacagcaagaacgacccggccaaggcggccgccagctttcaggcccaggtcatcgtgctccaccaccccggccagatcaacccgggctacacccccgtgctggactgccacacggcccacatcagctgcaagttcgcggacctggagaagaggctggacaggaggtcgggcaaggctctggaggactccccgaagagcatcaagtcgggcgacgccgctatcgtgaggatggagccgagcaagcccatgtgcgtcgaggcgttcatcgagtacccgcccctcggcaggttcgccgtgagggacatgaagcagaccatcgcggtgggcgtcatcaaggccgtcgagaagaaggaggcgggcggcaaggtgacgaagtcggctcagaaggccgcggctaagaaggacgagctgtgataggttaacctagacttgtccatcttctggattggccaacttaattaata

SEQ ID NO: 7 - protein, 596 aa, Eimeria maxima
MTRAAALAGVLALAAAGSSLALPTVLDTTTGTQVEWTETPLDTTEVTMGEMGSTTSGTTPTSTGVRMMEAETTTPSTPEAPQQQQQMPQPQPQPQQTTPVPEAVLEAIMQEMQNIFRSSLVPGWDTVGTAADAVRQIVTRVRERLTGPLMMTEMDTGLGRTGPLSTTGATGATTGPVAALRGVTNDFLREIMIQEAVLETLWAVVRDAQERPWLVNEQEALHAATADAVQGFLGRMHDRLRATGFSEEEVMRLLPRSRNGGCTRTGGLFDQCNDAPPSRLLGKRMYSTGYYGYGYPSYYSYGYSYPAYSHYPVSYPYYGYSWGPSYYYGSGYYGKHGYKYGHYYRRLAEQEPRPVMPPAAATAAANLRAAAAAAAEVPPPPPPAAVPPPPPAAAAGTPAMMPPPMMGVEEPVPFRSLYPSYSWSYPAYTRVSPSYSYYTPSYSSSYYYPRYNYAYNYPLYSDYSWYDYSYPLAYSSYSSYPLSYSSYSYPLSYTYPSAFYRRLEVPDLTTTTTTHHEQQQQQQQESTTTAVPTETITTPSTRNTHSSSLRRVGERYEPITPTQRTFYNNTEGTNNPVYTPENLTEDEPQTVWETYN

SEQ ID NO: 8 - DNA, 1856 bp, synthetic construct
  source 1..1856: mol_type = other DNA
accatggcgaacaagcacctgagccttagcctcttcctcgtgctcctgggcctctccgcctccctcgcctccggcgtcctgcctactgtgctcgacacgaccacgggcacccaggtggagtggacggagacgccgctcgacaccacggaggtgactatgggcgagatgggctccaccacgtctggcaccacgcccacctccacgggcgtccgcatgatggaggccgagacgacgaccccttccaccccggaggccccgcagcagcagcagcagatgccccagccacagccccagccacagcagaccacgccggtgcccgaggcggtcctcgaggccatcatgcaggagatgcagaacatcttcaggtccagcctggtgcccggctgggacaccgtgggcacagcggccgacgccgtgcgccagatcgtgacgcgcgtcagggagaggctcacgggccccctgatgatgacggagatggacaccggtctcggcaggacgggccccctgtccaccaccggcgccacaggcgctaccaccggccccgtggccgcgctcaggggcgtcacgaacgacttcctgcgcgagatcatgatccaggaggcggtgctggagacgctgtgggccgtggtcagggacgctcaggagcgcccgtggctggtgaacgagcaggaggccctgcacgctgccaccgccgacgccgtccagggcttccttggcaggatgcacgaccggctcagggctacgggcttcagcgaggaggaggtcatgaggctcctgccgaggtcccgcaacggcggctgcacgcgcaccggcggcctcttcgaccagtgcaacgacgccccgccaagcaggctcctgggcaagcgcatgtactccaccggctactacggctacggctacccctcctactacagctacggctactcctacccggcctacagccactaccccgtgtcctacccgtactacggctactcctggggcccgagctactactacggctctggctactacggcaagcacggctacaagtacggccactactaccgcaggctcgccgagcaggagccgaggcccgtcatgccgccagccgctgcgaccgccgccgcgaacctgcgcgccgccgctgccgccgctgccgaggtgccgccaccgccaccgccagctgccgtcccgcccccaccgccagcagcggccgctggcaccccggcgatgatgccgccgccaatgatgggggtggaggagcccgtcccattccgcagcttgtacccctcctacagctggagctaccccgcttacacgcgcgtgagcccctcctacagctactacaccccgagctactccagctcctactactaccccaggtacaactacgcctacaactacccgctctactccgactacagctggtacgactacagctaccccctggcctacagctcctacagctcctacccgctctcctacagctcctactcctaccccctgagctacacctacccgtcggccttctaccgcaggctggaggtgcccgacctgaccacgaccacgaccacgcaccacgagcaacaacagcagcagcagcaggagagcaccaccacggcggtgccaaccgagacgatcaccaccccgagcacgcgcaacacgcactccagctccctccgcagggtgggcgagcgctacgagcccatcacgccgacccagcgcaccttctacaacaacacggagggcaccaacaacccggtctacacgcccgagaacctgaccgaggatgagccgcagactgtgtgggagacctacaactgataggttaacctagacttgtccatcttctggattggccaacttaattaatc

SEQ ID NO: 9 - DNA, 1868 bp, synthetic construct
  source 1..1868: mol_type = other DNA
accatggcgaacaagcacctgagccttagcctcttcctcgtgctcctgggcctctccgcctccctcgcctccggcgtcctgcctactgtgctcgacacgaccacgggcacccaggtggagtggacggagacgccgctcgacaccacggaggtgactatgggcgagatgggctccaccacgtctggcaccacgcccacctccacgggcgtccgcatgatggaggccgagacgacgaccccttccaccccggaggccccgcagcagcagcagcagatgccccagccacagccccagccacagcagaccacgccggtgcccgaggcggtcctcgaggccatcatgcaggagatgcagaacatcttcaggtccagcctggtgcccggctgggacaccgtgggcacagcggccgacgccgtgcgccagatcgtgacgcgcgtcagggagaggctcacgggccccctgatgatgacggagatggacaccggtctcggcaggacgggccccctgtccaccaccggcgccacaggcgctaccaccggccccgtggccgcgctcaggggcgtcacgaacgacttcctgcgcgagatcatgatccaggaggcggtgctggagacgctgtgggccgtggtcagggacgctcaggagcgcccgtggctggtgaacgagcaggaggccctgcacgctgccaccgccgacgccgtccagggcttccttggcaggatgcacgaccggctcagggctacgggcttcagcgaggaggaggtcatgaggctcctgccgaggtcccgcaacggcggctgcacgcgcaccggcggcctcttcgaccagtgcaacgacgccccgccaagcaggctcctgggcaagcgcatgtactccaccggctactacggctacggctacccctcctactacagctacggctactcctacccggcctacagccactaccccgtgtcctacccgtactacggctactcctggggcccgagctactactacggctctggctactacggcaagcacggctacaagtacggccactactaccgcaggctcgccgagcaggagccgaggcccgtcatgccgccagccgctgcgaccgccgccgcgaacctgcgcgccgccgctgccgccgctgccgaggtgccgccaccgccaccgccagctgccgtcccgcccccaccgccagcagcggccgctggcaccccggcgatgatgccgccgccaatgatgggggtggaggagcccgtcccattccgcagcttgtacccctcctacagctggagctaccccgcttacacgcgcgtgagcccctcctacagctactacaccccgagctactccagctcctactactaccccaggtacaactacgcctacaactacccgctctactccgactacagctggtacgactacagctaccccctggcctacagctcctacagctcctacccgctctcctacagctcctactcctaccccctgagctacacctacccgtcggccttctaccgcaggctggaggtgcccgacctgaccacgaccacgaccacgcaccacgagcaacaacagcagcagcagcaggagagcaccaccacggcggtgccaaccgagacgatcaccaccccgagcacgcgcaacacgcactccagctccctccgcagggtgggcgagcgctacgagcccatcacgccgacccagcgcaccttctacaacaacacggagggcaccaacaacccggtctacacgcccgagaacctgaccgaggatgagccgcagactgtgtgggagacctacaacaaggacgagctgtgataggttaacctagacttgtccatcttctggattggccaacttaattaatc

(19) United States
(12) Patent Application Publication
(10) Pub. No.: US 2024/0417744 A1
(43) Pub. Date: Dec. 19, 2024

(54) EXPRESSION OF EIMERIA SEQUENCES IN PLANTS AND PLANT PRODUCED VACCINE FOR SAME

(71) Applicant: Mazen Animal Health Inc., Ames, IA (US)

(72) Inventors: John Howard, San Luis Obispo, CA (US); Erin Egelkrout, San Luis Obispo, CA (US); Joan Schrader, Ames, IA (US)

(73) Assignee: Mazen Animal Health Inc., Ames, IA (US)

(21) Appl. No.: 18/756,402

(22) Filed: Jun. 27, 2024

Related U.S. Application Data
(63) Continuation-in-part of application No. PCT/US2022/082240, filed on Dec. 22, 2022.
(60) Provisional application No. 63/266,112, filed on Dec. 29, 2021.

(51) Int. Cl. C12N 15/82; A61K 39/00; A61K 39/002; A61P 33/02

(52) U.S. Cl. CPC C12N 15/8258; A61K 39/002; A61P 33/02; A61K 2039/54; A61K 2039/552

(57) ABSTRACT

Vaccines and methods of expressing a polypeptide of Eimeria are provided in which a protective response to Eimeria is produced when administered to an animal. The vaccine provides for expression of Eimeria vaccine proteins 3-1e, Gam82, and/or EF-1a polypeptide in a plant or plant part, linked to a promoter preferentially directing expression to embryo tissue of the plant or plant part. Further embodiments provide that the polypeptide may be targeted to the apoplast/cell wall or the endoplasmic reticulum. Increased expression levels in the plant or plant part are obtained. The plant or plant materials in an embodiment may be orally administered.

### CROSS REFERENCE TO RELATED APPLICATIONS

This application is a continuation-in-part of International Application No. PCT/US2022/082240, filed Dec. 22, 2022, which claims priority under 35 U.S.C. § 119 to provisional patent application U.S. Ser. No. 63/266,112, filed Dec. 29, 2021, each of which, is herein incorporated by reference in its entirety, including without limitation, the specification, claims, and abstract, as well as any figures, tables, appendices, or drawings thereof.

### STATEMENT REGARDING FEDERALLY SPONSORED RESEARCH

This invention was made with Government support under SBIR Grant #2019-33610-29782, Production of a Candidate Vaccine for Avian Coccidiosis in maize, awarded by USDA-National Institute of Food and Agriculture. The Government has certain rights in this invention.

### REFERENCE TO SEQUENCE LISTING SUBMITTED ELECTRONICALLY

The instant application contains a Sequence Listing which has been submitted electronically in XML formatted and is herein incorporated by reference in its entirety. Said XML copy, created on Dec. 13, 2022, is named “P13608US01_SequenceListing.xml” and is 18,077 bytes in size.

### BACKGROUND

Coccidiosis, an infectious disease of poultry, swine, and cattle caused by apicomplexan protozoan parasites presents problems worldwide. In particular, poultry health and welfare is threatened by several pathogens and protozoan parasites of the genus Eimeria. Infection with Eimeria, which invade and replicate within gut epithelial cells, can compromise chicken welfare and require costly treatments. The expense of these treatments, combined with the losses caused by infection, are estimated to cost the global chicken industry more than $2 billion every year¹. Eimeria infection has also been demonstrated to exacerbate the outcome of exposure to other pathogens such as Clostridium perfringens, combining to cause necrotic enteritis².

Seven species of Eimeria are recognized to infect chickens and these vary in their fecundity, pathogenicity, and location of replication within the gut³. In addition, there are three circulating strains which cause the majority of infections, Eimeria acervulina, Eimeria tenella, and Eimeria maxima. Although immunization of chickens with live Eimeria oocysts is effective and has been the basis of live oral coccidiosis vaccines for almost seventy years, chickens must be vaccinated with oocysts from each Eimeria species to be fully protected⁴. The requirement for a live vaccine to include controlled doses of oocysts for all pathogenic species of Eimeria, and in some cases multiple strains of Eimeria maxima, makes vaccine manufacture logistically demanding as all vaccine lines must be propagated separately in chickens under stringent specific pathogen free conditions. Another important consideration is that fecal-oral recycling of vaccine parasites is required to generate levels of protective immunity that are sufficient to protect chickens against pathogenic challenge by most Eimeria species⁵. A recombinant vaccine that is protective against multiple Eimeria species is therefore highly desirable and this should contain multiple antigens, derived from different parasite species and lifecycle stages.

Control of Eimeria in commercial chicken production currently relies on a combination of anticoccidial drugs (chemicals and antibiotics) and vaccination using formulations of live virulent or attenuated Eimeria species. Mass administration of anticoccidial drugs has long been employed as a method of control, however drug resistance is now widespread, no new chemical control measures are in development, and public/legislative pressures to reduce use of antibiotics in livestock has increased. In addition, existing vaccines are expensive and can cause detrimental stress to the animals. Addressing this need, plant-produced vaccines can supply large amounts of vaccine proteins while minimizing production costs and equipment use. For example, maize-produced vaccines are cost-effective, heat-stable, and suitable for direct feeding to a variety of animals.

The present inventors therefore sought to produce, optimize, and characterize maize lines expressing high levels of 3-1e, EF-1 alpha, and Gam82 to induce an immune response to one or more of Eimeria acervulina, Eimeria tenella, and Eimeria maxima. A secondary goal was to demonstrate that bioencapsulated Eimeria antigens in maize grain when delivered orally provide an efficacious vaccine. The commercial target of the present invention is a vaccine providing greater than 85% protection in chickens against an Eimeria spp. challenge. Successful completion of the work will lead to future development of a low-cost, heat-stable, effective oral vaccine for avian coccidiosis that should reduce reliance on antibiotics and economic losses related to this disease.

### BRIEF SUMMARY

A vaccine for Eimeria is provided which is produced from a plant. Provided are vaccines and methods of producing a protective response to Eimeria in an animal. When the plant or plant product is orally administered to an animal, a protective response is observed. Embodiments provide orally administering a plant or plant product including at least one Eimeria vaccine protein expressed at levels of at least 10 mg/kg in seed of said plant. Eimeria vaccine proteins include 3-1e, Gam82, and/or EF-1a. Embodiments provide a promoter preferentially directing expression to embryo tissue of a plant. Further embodiments provide for targeting expression of the Eimeria vaccine proteins to the apoplast or endoplasmic reticulum. Additional embodiments provide the construct and plasmids for expression of the same at high levels in plants. As noted, the plant product may comprise at least one, two, three, or more Eimeria antigens and offer protection against multiple Eimeria species. That is, an Eimeria antigen derived from one species can offer protection against multiple Eimeria species. In another embodiment, different Eimeria antigens may be produced in multiple plants and mixed or provided together in animal feed. Alternatively, plants expressing at least one of the Eimeria antigens may be crossed with plants expressing another one or more Eimeria antigens to provide plants expressing multiple Eimeria antigens.

### BRIEF DESCRIPTION OF THE DRAWINGS

FIG. 1 is a graphic showing a schematic representation of Eimeria antigen expression constructs. Four plasmid constructs were designed to variously express Eimeria antigens 3-1e, Gam82, and/or EF-1 polypeptides (referred to collectively as “Eimeria vaccine proteins”) in maize. The four constructs include: construct EIA (expressing 3-1e and Gam82), construct EIB (expressing EF-1a), construct a (expressing 3-1e and EF-1a), and construct EIE (expressing Gam82). EIA and EIB are targeted to the apoplast/cell wall and EID and EIE to the endoplasmic reticulum. Cellular/organelle-targeting sequences (BAASS:cell wall/apoplast) were included in all constructs. C-terminus sequences (KDEL:ER) were included in the codon-optimized EID and EIE constructs. Embryo preferred promoter (Pr44:seed embryo) was used to drive high gene transcription and subsequent high yield of protein production in all constructs. The untranslated 3′ region of a potato protease inhibitor II (pinII) was also added to all constructs as a transcriptional termination element to enhance mRNA stability.

FIG. 2 is a Western Blot of seed grain expression of 3-1e in constructs EIA and EID. Seeds were harvested and analyzed by Western blots to detect for 3-1e. Each lane represents seed from one plant from an independent insertion event in either EIA or EID. Lane 1 is the 3-1e standard showing a specific band at the expected MW of ˜27 kD. Notably, bands running larger than expected in Lane 1 are due to 3-1e aggregation and/or post-translational modifications. Lane 2 is a standard ladder. Lane 3 (Hill) is an untransformed negative control. Lanes 4-7 represent EIA constructs, with lanes 4, 5, and 7 showing a specific band at the expected MW of ˜27 kD. Lanes 8-10 represent EID constructs with lanes 8 and 10 showing a specific band at the expected MW of ˜27 kD.

FIG. 3 is a Western Blot of seed grain expression of EF-1a in constructs EIB and EID. Seeds were harvested and analyzed by Western blots to detect for EF-1a. Each lane represents seed from one plant from an independent insertion event in either EIB or EID. Lane 1 is the EF-1a standard and Lane2 is a standard ladder. Lane 3 (Hill) is an untransformed negative control. Lanes 4-7 represent EIB constructs and lanes 8-10 represent EID constructs. Lanes 4-10 show specific bands at the expected MW of ˜51 kD. Bands may appear to run larger due to post-translational modifications.

FIG. 4 is a Western Blot of seed grain expression of Gam82 in constructs EIA and EIE. Seeds were harvested and analyzed by Western blots to detect for Gam82. Each lane represents seed from one plant from an independent insertion event in either EIA or EIE. Lanes 4-7 represent EIA constructs and lanes 8-10 represent EIE constructs. Lane 1 is the Gam82 standard showing a specific band at the expected MW of ˜67 kD (see arrow). Lane 2 is a standard ladder. Lane 3 (Hill) is an untransformed negative control. Lanes 4-10 show specific bands at the expected MW of ˜67 kD. Bands may appear to run larger due to post-translational modifications.

FIG. 5 is a plasmid map of EIA pr44-BAASS:3-1e pr44-BAASS:Gam82.

FIG. 6 is a plasmid map of EIB pr44-BAASS:EF-1a.

FIG. 7 is a plasmid map of EID pr44-BAASS:3-1e KDEL pr44-BAASS:EF-1a KDEL.

FIG. 8 is a plasmid map of EIE pr44-BAASS:Gam82 KDEL.

FIGS. 9A-C depict results from a proof of concept study for an Eimeria vaccine in broiler chickens. FIG. 9A depicts lesion score results for E. acervulina in four treatment groups (24 birds per group). A significant vaccine effect was observed for the high dose group. FIG. 9B depicts adjusted feed conversion 12 days post-challenge (study days 27-33) in four treatment groups (48 birds per group). The high dose vaccine group had significantly better feed conversion than the positive control and low dose vaccine groups. FIG. 9C depicts the percent reduction in oocyst output 6 days post-challenge in four treatment groups. The results show a reduction in output for both high and low dose vaccine groups.

### DESCRIPTION

Eimeria offers several advantages as a model parasitic pathogen for successful vaccine development. The present inventors have produced, optimized, and characterized maize lines expressing high levels of Eimeria proteins in order to advance the development of a vaccine. Eimeria antigens were produced together in the kernels of a single maize line, and were used to induce an immune response in animal subjects to Eimeria acervulina, Eimeria tenella, and Eimeria maxima. The use of plant-produced proteins and the simultaneous use of multiple Eimeria antigens is anticipated to result in a greater protective effect than prior studies. This simple and cost-effective method of production and administration of Eimeria vaccine proteins greatly advances vaccine development in chickens and other animals.

A preferred embodiment of the present invention uses maize grain as a basis for the production of a subunit vaccine based on Eimeria antigens 3-1e, Gam82, and EF-1a. In embodiments, subunit vaccines may be based on immunogens/antigens derived from avian protozoan antigens, including antigens derived from Eimeria acervulina, such as 3-1e (Lillehoj, H S et al., 2005; Ding, X. et al., 2004), antigens from Eimeria maxima, such as Gam82 (Antigens from Belli, S. et al., 2004), and antigens from Eimeria tenella, such as Eimeria tenella Elongation Factor-1a (EF-1a) (Wu, S. et al., 2004; Pogonka, T. et al., 2003). High expression levels of at least 1 mg/kg of whole seed are obtained. An embodiment provides for an average range of about 10-600 mg/kg. Further embodiments provide for expression at 11 mg/kg, 12 mg/kg, 13, 14, 15, 16, 17, 18, 19, 20, 25, 30, 35, 40 mg/kg of whole seed or more or amounts in-between.

Oral administration of the plant, plant part or a product produced from the plant part, such as a seed, grain, flour or other edible composition comprising the plant, plant part or product produced therefrom comprising Eimeria proteins (e.g., 3-1e, Gam82, and/or EF-1a) can result in protection against challenge for the subject animal. The subject animal may or may not produce detectable antibodies in response, but the animal will have decreased morbidity or mortality resulting from administration of the vaccine, such that upon exposure to disease challenge, the animal is able to combat the infection. The compositions of the invention can also induce a surprising serum antibody response as well as a mucosal response. The serum antibody response in an embodiment is within the range of two to 100-fold more than the control. In another embodiment the response can be 5 times, 10 times, 15 times, 20 times, 25 times, 30 times, 35 times, 40 times, 45 times, 50 times, 55 times, 60 times, 65 times, 70 times, 75 times, 80 times, 85 times, 90 times, 95 times or more greater than control animals not receiving vaccination, or amounts in-between.

As used herein, the term “animal” or “subject” or “subject animal” is intended to include chickens and other animals including human beings.

As used herein, the terms nucleic acid or polynucleotide refer to deoxyribonucleotides or ribonucleotides and polymers thereof in either single- or double-stranded form. The sequence used to make the vaccine may be obtained from any source, such as a biological source in isolating from a biological sample or can refer to a sequence synthetically produced based upon the sequence obtained from the sample. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally occurring nucleic acid molecules, which can be isolated from a cell, as well as synthetic molecules, which can be prepared, for example, by methods of chemical synthesis or by enzymatic methods such as by polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

Nucleic acids employed here include those that encode an entire polypeptide as well as those that encode a subsequence of the polypeptide or produce a fragment that provides a protective response. For example, nucleic acids that encode a polypeptide which is not full-length but nonetheless has protective activity against Eimeria. The invention includes not only nucleic acids that include the nucleotide sequences as set forth herein, but also nucleic acids that are substantially identical to, correspond to, or substantially complementary to, the exemplified embodiments. For example, the invention includes nucleic acids that include a nucleotide sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%, 94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The nucleotide sequence may be modified as described previously, so long as any polypeptide produced is capable of inducing the generation of a protective response. A protective response includes a robust serum antibody response in an animal.

The nucleic acids can be obtained using methods that are known to those of skill in the art. Suitable nucleic acids (e.g., cDNA, genomic, or subsequences) can be cloned, or amplified by in vitro methods such as the polymerase chain reaction (PCR) using suitable primers, the ligase chain reaction (LCR), the transcription-based amplification system (TAS), or the self-sustained sequence replication system (SSR). A wide variety of cloning and in vitro amplification methodologies are well-known to persons of skill in the art. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif (Berger); Sambrook et al. (2001) Molecular Cloning—A Laboratory Manual (Third ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C& EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Nucleic acids or subsequences of these nucleic acids, can be prepared by any suitable method as described above, including, for example, cloning and restriction of appropriate sequences.

“Codon optimization” can be used to optimize sequences for expression. This is defined as modifying a nucleic acid sequence for enhanced expression in the cells of the recombinant host by replacing at least one, more than one, or a significant number, of codons of the native sequence with codons that are more frequently or most frequently used in the recombinant host. Various species exhibit particular bias for certain codons of a particular amino acid.

As used herein, a “polypeptide” refers generally to peptides and proteins. In certain embodiments the polypeptide may be at least two, three, four, five, six, seven, eight, nine or ten or more amino acids or more or any amount in-between. A peptide is generally considered to be more than fifty amino acids. The terms “fragment,” “derivative” and “homologue” when referring to the polypeptides according to the present invention, means a polypeptide which retains essentially the same biological function or activity as said polypeptide, that is, act as an antigen and/or provide treatment for and/or protection against disease. Such fragments, derivatives and homologues can be chosen based on the ability to retain one or more of the biological activities of the polypeptide, that is, act as an antigen and/or provide treatment for and/or protection against the pathogen. The polypeptide vaccines of the present invention may be recombinant polypeptides, natural polypeptides or synthetic polypeptides, preferably recombinant polypeptides. One skilled in the art appreciates that it is possible that the protective polypeptide may be expressed by the gene in the host cells and the plant composition administered to the animal or extracted from the plant prior to administration.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques. Many of these techniques are described in detail below. Notably, the phrase Eimeria protein(s) is used interchangeably with Eimeria antigen(s) herein.

The methods include amino acids that include an amino acid sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%, 94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The sequence may be modified as described previously, so long as the polypeptide is capable of inducing the generation of a protective response.

The variant proteins used in the present methods can be attached to heterologous sequences to form chimeric or fusion proteins. Such chimeric and fusion proteins comprise a variant protein fused in-frame to a heterologous protein having an amino acid sequence not substantially homologous to the variant protein. The heterologous protein can be fused to the N-terminus or C-terminus of the variant protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present methods further provide functional fragments of the nucleic acid molecules and polypeptides including variant proteins of the polypeptide, in addition to proteins and peptides that comprise and consist of such fragments, provided that such fragments act as an antigen and/or provide treatment for and/or protection against Eimeria.

As used herein, the term “subunit” refers to a portion of the microorganism which provides protection and may itself be antigenic, i.e., capable of inducing an immune response in an animal. The term should be construed to include subunits which are obtained by both recombinant and biochemical methods.

In one embodiment, a method of identifying protective sequences of the virus or nucleic acids that elicit protection is provided. This method also includes fragments, derivatives, or homologs of the nucleic acid molecule. In one aspect, the method comprises administering to a test animal such sequences. The test and control animals are subsequently challenged with an infectious amount of a microorganism that causes the disease. Various methods and techniques for determining whether protection is provided are known to those skilled in the art, including but not limited to, observing a difference between the test and control animal in the symptoms of the disease, for example. A decrease in any of the symptoms observed in the test animal compared to the control animal indicates that protective molecule(s) provide a degree of protection against disease. Similar symptoms or an increase in any of the symptoms observed in the test animal compared to those observed in the control animal indicate that the protective molecule(s) do not provide protection.

In another aspect, determining whether the molecules provided protection against Eimeria includes determining the presence or absence of challenge disease in the test animal by electron microscopy or antibody or assays such as the fluorescent focusing neutralizing (FFN) test or Western blot assay may be used. PCR methods may be used to determine if the protective molecule is present. Northern blotting can detect the presence of diagnostic sequences. In another aspect, an ELISA or similar assay, such as a hemagglutinin inhibition assay are the types of many varied assays that can determine if the protective molecule is effective. The ELISA or enzyme linked immunoassay has been known since 1971. In general, antigens solubilized in a buffer are coated on a plastic surface. When serum is added, antibodies can attach to the antigen on the solid phase. The presence or absence of these antibodies can be demonstrated when conjugated to an enzyme. Adding the appropriate substrate will detect the amount of bound conjugate which can be quantified. A common ELISA assay is one which uses biotinylated anti-(protein) polyclonal antibodies and an alkaline phosphatase conjugate. For example, an ELISA used for quantitative determination of protein levels can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate.

Clearly, many such methods are available to one skilled in the art to ascertain if the molecule provides protection and provides protection at the levels administered to the animal.

The nucleic acid molecule, polypeptide or fragment thereof, when administered to the subject animal produces a protective response to Eimeria. A protective response is elicited in the animal. The subject animal may or may not produce antibodies in response, but the animal will have decreased morbidity or mortality resulting from administration of the vaccine, and as described further herein. The terms “protecting”, “protection”, “protective immunity” or “protective immune response,” as used herein, are intended to mean that the host subject animal mounts an active immune response to the vaccine or polypeptides of the present invention, such that upon exposure to disease challenge, the subject animal is able to combat the infection. Thus, a protective immune response will decrease the incidence of morbidity and mortality from exposure to the microorganism among a host animal. The subject animal will be protected from subsequent exposure to the disease-causing agent. In an embodiment, the animal may be protected by treating the animal which has already been exposed to the disease-causing agent by administration of the vaccine or polypeptide after such exposure. In such an instance there is also shown to be a lessening of morbidity and mortality. Those skilled in the art will understand that in a commercial animal setting, the production of a protective immune response may be assessed by evaluating the effects of vaccination on the herd as a whole, e.g., there may still be morbidity and mortality in a minority of vaccinated animals. Furthermore, protection also includes a lessening in severity of any gross or histopathological changes and/or of symptoms of the disease, as compared to those changes or symptoms typically caused by the isolate in similar animals which are unprotected (i.e., relative to an appropriate control). Thus, a protective immune response will decrease the symptoms of the disease compared to a control animal.

A “construct” is a package of genetic material inserted into the genome of a cell via various techniques. A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. The term “vector” includes both viral and nonviral means for introducing the nucleic acid into a cell in vitro, ex vivo, or in vivo. Viral vectors include alphavirus, retrovirus, adeno-associated virus, pox, baculovirus, vaccinia, herpes simplex, Epstein-Barr, rabies virus, vesicular stomatitis virus, and adenovirus vectors. Non-viral vectors include, but are not limited to plasmids, liposomes, electrically charged lipids (cytofectins), DNA- or RNA protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A nucleic acid molecule is introduced into a cell when it is inserted in the cell. A cell has been “transfected” by exogenous or heterologous DNA or RNA when such DNA or RNA has been introduced inside the cell.

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene; and DNA elements that control the processing of transcripts, such as transcription termination/polyadenylation sequences. It also can contain such sequences as are needed for the eventual integration of the vector into the host chromosome. By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

A nucleic acid molecule may be used in conjunction with its own or another promoter. In one embodiment, a selection marker and a nucleic acid molecule of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive a nucleic acid molecule of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any host-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed.

A promoter can additionally comprise other recognition sequences generally positioned upstream or 5′ to the TATA box, referred to as upstream promoter elements, which influence the transcription initiation rate. It is recognized that having identified the nucleotide sequences for a promoter region, it is within the state of the art to isolate and identify further regulatory elements in the 5′ region upstream from the particular promoter region identified herein. Thus, the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular tissue. When referring to preferential expression, what is meant is expression at a higher level in the particular tissue than in other tissue. Examples of these types of promoters include embryo preferred expression such as that provided by the phaseolin promoter (Bustos et al. (1989) The Plant Cell Vol. 1, 839-853). For dicots, embryo-preferred promoters include, but are not limited to, bean β-phaseolin, napin, β-conglycinin, soybean lectin, cruciferin, and the like. For monocots, embryo-preferred promoters include, but are not limited to, maize 15 kD zein, 22 kD zein, 27 kD zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where embryo-preferred promoters from end1 and end2 genes are disclosed. Seed-preferred promoters also include those promoters that direct gene expression predominantly to specific tissues within the seed such as, for example, the endosperm-preferred promoter of γ-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to a seed or an embryo preferred promoter is meant that it expresses an operably linked sequence to a higher degree in seed or embryo tissue than in other plant tissue. It may express during seed or embryo development, along with expression at other stages, may express strongly during seed or embryo development and to a much lesser degree at other times. The globulin promoter shown here preferentially expresses to the embryo of the plant. Globulin-1 is the most abundant protein in maize embryos and is a vicilin-like storage protein encoded by the globulin-1 gene. See, e.g., Liu et al. (1992) MNL Vol. 22: 108-109. As noted in Belanger et al. (1991) globulins are storage proteins recognized as comprising 10-20% of the maize embryo protein and Globulin 1 is one of the most abundant proteins, encoded by the globulin-1 gene. Belanger, F. C. and Kriz, A. L. (1991) “Molecular basis for allelic polymorphism of the maize globulin-1 gene” Genetics 129, 863-872. The two most abundant proteins in maize embryos are saline-soluble, water-insoluble globulins, one being a 63,000 Da molecular weight protein encoded by the globulin-1 gene, the other a 45,000 Da molecular weight protein encoded by the globulin-2 gene. See. e.g, Kriz (1989) Biochem Genet. 27(3-4):238-51. Where a null allele is present no Globulin 1 protein is produced. Belanger et al. (1991), supra. Belanger et al. note that the protein is readily detected in a Coomassie-stained gel of protein extracts from embryos and several alleles have been recognized. Belanger et al. (1991), at 865. One skilled in the art appreciates that nucleic acid molecules that encode the Globulin 1 protein are well known and readily identified using techniques available to one skilled in the art and as discussed here, including, by way of example without limitation, comparison to known sequences, preparation of a library and screening with a probe, antibody binding, using Northern, Southern or Western blots, among the many avenues available. The promoter of a globulin-1 encoding gene may be used in plants to express operably linked nucleic acid molecules in a plant. Examples, without intending to be limiting, of globulin promoters include the 1.45 kb maize globulin-1 promoter plus untranslated leader described by Belanger and Kriz, 1991, supra and GenBank accession L22344.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A cell containing an inducible regulatory element may be exposed to an inducer by externally applying the inducer to the cell or plant such as by spraying, watering, heating or similar methods.

Any inducible promoter can be used. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)); Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns, etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall and/or apoplast use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL. This sequence (Lys-Asp-Glu-Leu) contains the binding site for a receptor in the endoplasmic reticulum. (Munro et al., (1987) “A C-terminal signal prevents secretion of luminal ER proteins.” Cell. 48:899-907. There are a wide variety of endoplasmic reticulum retention signal sequences available to one skilled in the art and the KDEL sequence is one example. Another example is HDEL (His-Asp-Glu-Leu). See, for example, Kumar et al. which discuses methods of producing a variety of endoplasmic reticulum proteins. Kumar et al. (2017) “prediction of endoplasmic reticulum resident proteins using fragmented amino acid composition and support vector machine” Peer J. doi: 10.7717/peerj.3561.

Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) PlantMolec. Biol. 14:357-368).

The termination region can be native with the DNA sequence of interest or can be derived from another source. Convenient termination regions are available from the Ti-plasmid of A. tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp7831-7846 (nos)). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Many variations on the promoters, selectable markers, signal sequences, leader sequences, termination sequences, introns, enhancers and other components of the vector are available to one skilled in the art.

Plant is used broadly herein to include any plant at any stage of development, or to part of a plant, including a plant cutting, a plant cell culture, a plant organ, a plant seed, and a plantlet. In other words, the term plant refers to the entire plant or plant material or plant part or plant tissue or plant cell including a collection of plant cells. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, plants may be regenerated from the tissue cultures.

A variety of plant species may be used, including any plant species, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis) algae, or Lemnoideae (aka Duckweed). An embodiment provides the plant is maize.

Various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed. Notably, the transformation vector comprising the sequence operably linked to a heterologous nucleotide sequence in an expression cassette, can also contain at least one additional nucleotide sequence for a gene to be cotransformed into the organism. Alternatively, the additional sequence(s) can be provided on another transformation vector.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993). Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In an embodiment the Hill maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the 5,591,616 patent, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the 5,591,616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase and are harvested at a density of no more than OD₆₀₀=0.5, preferably between 0.2 and 0.5. The bacteria are then centrifuged to remove the media and resuspended in the co-culture medium. Since Hill is used, medium preferred for Hill is used. This medium is described in considerable detail by Armstrong and Green (1985). The resuspension medium is the same as that described above. All further Hill media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity and becomes “reprogrammed” to have a new identity. Thus, the scutellum cells become embryogenic callus.

A transgenic plant may be produced that contains an introduced nucleic acid molecule encoding the polypeptide.

When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman (1995) Breeding Field Crops. AVI Publication Co., Westport Conn, 4^{th} Edit.). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detassling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Selection and propagation techniques described above can yield a plurality of transgenic plants that are harvested in a conventional manner. The plant or any parts expressing the recombinant polypeptide can be used in a commercial process, or the polypeptide extracted. When using the plant or part itself, it can, for example, be made into flour and then applied in the commercial process. Polypeptide extraction from biomass can be accomplished by known methods. Downstream processing for any production system refers to all unit operations after product synthesis, in this case protein production in transgenic seed (Kusnadi, A. R., Nikolov, Z. L., Howard, J. A., 1997. Biotechnology and Bioengineering. 56:473-484). For example, seed can be processed either as whole seed ground into flour or, fractionated and the germ separated from the hulls and endosperm. If germ is used, it is usually defatted using an extraction process and the remaining crushed germ ground into a meal or flour. In some cases, the germ is used directly in the process or the protein can be extracted (See, e.g., WO 98/39461). Extraction is generally made into aqueous buffers at specific pH to enhance recombinant protein extraction and minimize native seed protein extraction. Subsequent protein concentration or purification can follow.

The compositions and process described here are also for producing and administering a vaccine that protects an animal from Eimeria.

As used herein, the term “vaccine” refers to a pharmaceutical composition comprising at least one protective molecule, that induces protective response in a subject and possibly, but not necessarily, one or more additional components that enhance the activity of said active component. A vaccine may additionally comprise further components typical to pharmaceutical compositions. In another form, the immunologically active component of a vaccine may comprise appropriate elements of said organisms (subunit vaccines) whereby these elements are generated either by destroying the whole organism or the growth cultures of such microorganisms and subsequent purification steps yielding in the desired structure(s), or by synthetic processes induced by an appropriate manipulation of a suitable system such as, but not restricted to, bacteria, insects, mammalian, or other species, plus subsequent isolation and purification procedures or by induction of said synthetic processes in the animal needing a vaccine by direct incorporation of genetic material using suitable pharmaceutical compositions (polynucleotide vaccination). A vaccine may comprise one or simultaneously more than one of the elements described above.

The present vaccines may include a pharmaceutically acceptable carrier, excipient, carrier, stabilizer and/or diluent. Without intending to be limiting, examples include wetting agents and lubricating agents, preservative agents, lipids, stabilizers, solubilizers and emulsifiers. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. Examples of stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin).

It is possible to provide an adjuvant in the vaccine. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Immunostimulatory agents or adjuvants have been used for many years to improve the host immune responses to, for example, vaccines. The vaccines of the present invention may be used in conjunction with adjuvants, for example, lipopolysaccharides, aluminum hydroxide and aluminum phosphate (alum), saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes. Desirable characteristics of ideal adjuvants may include: (1) lack of toxicity; (2) ability to stimulate a long-lasting immune response; (3) simplicity of manufacture and stability in long-term storage; (4) ability to elicit both CMI and HIR to antigens administered by various routes; (5) synergy with other adjuvants; (6) capability of selectively interacting with populations of antigen presenting cells (APC); (7) ability to specifically elicit appropriate T-cell helper 1 (TH 1) or TH 2 cell-specific immune responses; and (8) ability to selectively increase appropriate antibody isotype levels (for example, IgA) against antigens. An adjuvant used with the present compositions and methods need not possess all these characteristics to be used.

As used herein, “immunogenically effective amount” refers to an amount, which is effective in reducing, eliminating, treating, preventing or controlling the symptoms of the infections, diseases, disorders, or condition.

The quantity to be administered depends on the subject to be treated, including, for example, the capacity of the immune system of the individual to mount a protective response. Suitable regimes for initial administration and booster doses are also variable but may include an initial administration followed by subsequent administrations. For example, it may be desirable to provide for an initial administration of the vaccine followed by additional doses. The need to provide an effective amount of the protective molecule will also need to be balanced with cost of providing higher amounts of the protective molecule. A cost-effective vaccine is one in which the cost of producing it is less than the value one can obtain from using it. Measurement and determination of efficacy of any of the compositions and vaccines of the invention may be accomplished by any of the many methods available to one skilled in the art.

In one embodiment, a straightforward and quick method can be to perform a Western blot analysis of a sample candidate vaccine composition to quantitate the amount of polypeptide or fragment that is present in the sample. In one embodiment, one compares the amount of polypeptide to a standard known to be effective with like polypeptides from other biotypes, and either prepares a vaccine where the level of polypeptide produced is at least at this standard or higher or may test the vaccine with a test animal.

The compounds described herein can be administered to a subject at therapeutically effective doses to prevent Eimeria-associated diseases. The dosage will depend upon the subject receiving the vaccine as well as factors such as the size, weight, and age of the subject.

The precise amount of immunogenic composition of the invention to be employed in a formulation will depend on the route of administration and the nature of the subject (e.g., age, size, stage/level of disease), and should be decided according to the judgment of the practitioner and each subject's circumstances according to standard clinical techniques. An effective immunizing amount is that amount sufficient to treat or prevent an Eimeria infectious disease in a subject, for example an avian subject.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response.

The immune response of the test subjects can be analyzed by various approaches such as: the reactivity of the resultant immune serum to the immunogenic conjugate, as assayed by known techniques, e.g., enzyme linked immunosorbent assay (ELISA), immunoblots, immunoprecipitations, virus neutralization, etc.; or, by protection of immunized hosts from infection by the pathogen and/or attenuation of symptoms due to infection by the pathogen in immunized hosts as determined by any method known in the art, for assaying the levels of an infectious disease agent, e.g., the viral levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

In addition, the therapeutics may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

The therapeutics can also be assessed by measuring the level of the molecule against which the antibody is directed in the animal model and/or human subject at suitable time intervals before, during, or after therapy. Any change or absence of change in the amount of the molecule can be identified and correlated with the effect of the treatment on the subject. The level of the molecule can be determined by any method known in the art.

Following vaccination of an animal to Eimeria using the methods and compositions of the present invention, any binding assay known in the art can be used to assess the binding between the resulting antibody and the particular molecule. These assays may also be performed to select antibodies that exhibit a higher affinity or specificity for the particular antigen. As one measure of vaccine potency, an ELISA can be performed on a sample collected from an individual vaccinated to determine whether antibodies to a vaccine comprising the sequence, a derivative, a homologue or a variant or fragment thereof generated anti-polypeptide antibodies. The individual's sample is measured against a reference anti-polypeptide antibody. Analysis of symptoms and measurement of animal weight gain also demonstrated lessening of impact of the disease in the presence of a particular dose. Fluorescent focused neutralization assay is still another assay to detect serum neutralizing antibodies and analyze effectiveness of a vaccine and a particular dose. When testing animals administered the vaccine, for example, measuring antibody response is also effective in determining efficacy of the vaccine. Sera may be collected and titer measured as the reciprocal of the maximal dilution at which hemagglutination is inhibited, as described in an example below. Other measurements post-administration of the vaccine can also be employed to determine effectiveness, whether pathological evaluation, isolation of the pathogen, measurement of symptoms, and overall health and weight gain of the subject.

Vaccine effectiveness may also be evaluated quantitatively (for example, a decrease in the percentage of diseased tissue as compared to an appropriate control group) or qualitatively (e.g., isolation of virus from blood, detection of virus antigen in a tissue sample by an assay method, etc.). The symptoms of the disease may be evaluated quantitatively (e.g., temperature/fever), semi-quantitatively (e.g., severity of distress), or qualitatively (e.g., the presence or absence of one or more symptoms or a reduction in severity of one or more symptoms). Clearly one skilled in the art has many different options available for measuring effectiveness of the vaccine. Protection periods of more than seven days after at least one challenge or exposure to the pathogenic microorganism have been achieved, and protection of at least two weeks, at least 20 days, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more, have been achieved using the invention. Such protection periods are also provided when using the invention with other animals. The protective response is also shown here in an embodiment to be specific to the disease as opposed to another disease, and thus demonstrates specific memory.

Standard methods can be used to administer the vaccine including intranasal, oral and/or parenteral (e.g., intramuscular) administration. For example, the Eimeria vaccine protein-containing vaccine can be administered intramuscularly one or more times. In another embodiment of the method, for example, the vaccine is administered orally one or more times. In an alternative embodiment, oral administration can be followed by and/or preceded by administration of the vaccine at least once, intramuscularly. The maize grain can be made into a food product and fed to the animal, thereby reducing cost and loss of antigen that can occur through further processing.

The following is provided by way of illustration without intending to be limiting of the scope of the invention. All references cited herein are incorporated herein by reference.

### EXAMPLES

### Example 1

Plant-produced proteins have shown great promise in that they can accumulate recombinant proteins at much higher levels than microbes for some recalcitrant proteins and are the least expensive source of proteins in general⁶⁻⁸. Not all plant systems, however, are created equal. There is a wide variation in the cost, scalability, agents that interfere with purification such as proteases, lignin and phenols, storage properties and safety with regard to allergenic, carcinogenic or toxin material in the host. These characteristics have been reviewed elsewhere⁷ and maize has emerged as one of the premier systems leading to recent commercialization of several recombinant proteins.

This is evidenced by high accumulation observed in several vaccine candidates. Reports of the accumulation of hepatitis B surface antigen (HBsAg) in edible plant tissue (other than maize) have varied dramatically with banana fruit being one of the lowest at 0.001 μg/gram fresh weight and potato being one of the highest at 8 μg/gram fresh weight^{9,10}. In a maize-based system developed by the present inventors, HBsAg has been expressed at >200 μg/g. This high level of antigen in the grain leads to a cost of the raw material below $0.01/dose even when accumulation is only 10 μg/g. However, purification costs can account for 90% of the product and is inversely proportional to the concentration in the biomass. Cost models have shown that levels as low as 10 μg/g may be economically feasible but for most cases levels of >50 mg/kg are targeted to keep purification costs low. This level is approximately 5-fold higher than what has been achieved in microbes. The present inventors have shown that the same maize-based system may be used to express Eimeria vaccine proteins at similarly high levels and at low cost.

Indeed, processed maize grain is cost-effective, heat-stable and suitable for direct feeding to animal subjects. Incorporation of subunit Eimeria vaccine proteins such as Eimeria 3-1e, Gam82, and E-1Fa into maize grain also allows for bioencapsulation, which protects the subunit vaccine proteins from degradation in the digestive system upon oral delivery to a subject. Further, bioencapsulated Eimeria proteins in maize grain leads to a more efficacious vaccine when delivered orally. For example, it is anticipated that the present invention will provide greater than 85% protection in chickens against an Eimeria spp. challenge.

Therefore, upon embarking on accumulating Eimeria antigens in maize to produce a candidate vaccine for Eimeria, several approaches to accumulate high levels were employed as described herein.

### Example 2

### Create Molecular Constructs

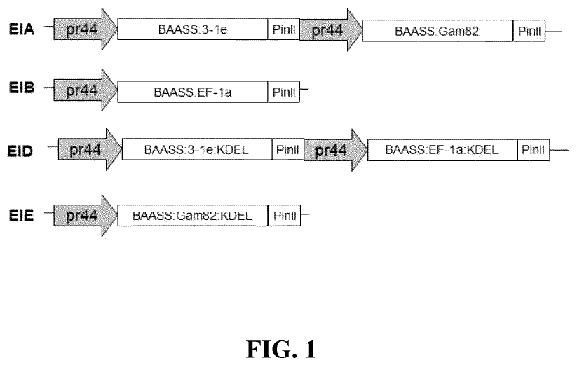

To develop an Eimeria vaccine, maize was transformed with four constructs coding for Eimeria vaccine proteins 3-1e polypeptide, Gam82 polypeptide, and/or EF-1 polypeptides (FIG. 1). The four constructs included: EIA (expressing 3-1e and Gam82), EIB (expressing EF-1a), EID (expressing 3-1e and EF-1a), and EIE (expressing Gam82). The sequence of 3-1e (GenBank AAD39362), Gam82 (GenBank XP_013333566), and EF-1a (GenBank XP_013234784) were optimized for maize codon usage and expressed in maize in a manner similar to our previously published work¹¹. The open reading frames are linked to embryo-preferred promoters in a manner similar to our previously published work^{12,13} that confers high expression with a potato protease inhibitor II (pinII) terminator sequence¹⁴. Notably the phrase “Eimeria vaccine proteins”, “Eimeria proteins”, and “Eimeria antigens” are used interchangeably herein. In addition, “EF-1a” and “EF-1 alpha” are used interchangeably herein.

Eimeria vaccine proteins 3-1e, EF-1 alpha, and/or Gam82 can have a targeting signal directed to the apoplast/cell wall using a well-characterized barley alpha-amylase (“BAASS”) targeting sequence, or a targeting signal directed to the endoplasmic reticulum (ER) using a KDEL C-terminal targeting signal sequence as described previously¹⁵. Both subcellular localization strategies result in high levels of expression. Indeed, the apoplast and endoplasmic reticulum have proven to be consistent cellular locations for accumulating antigens in maize grain.

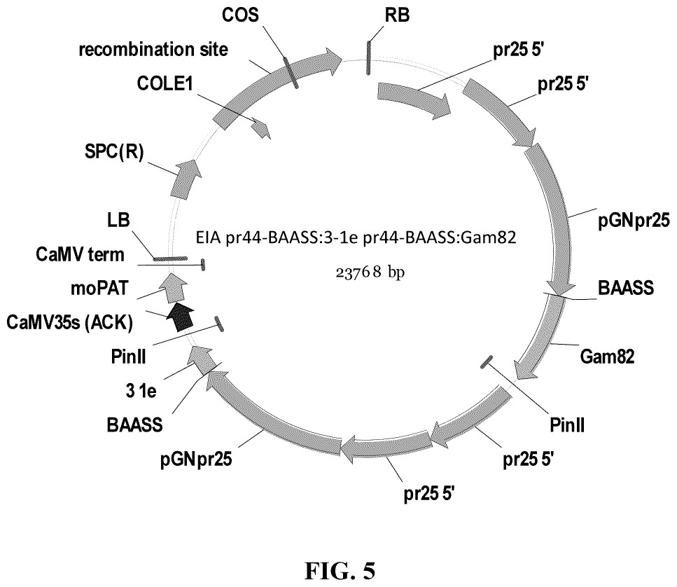

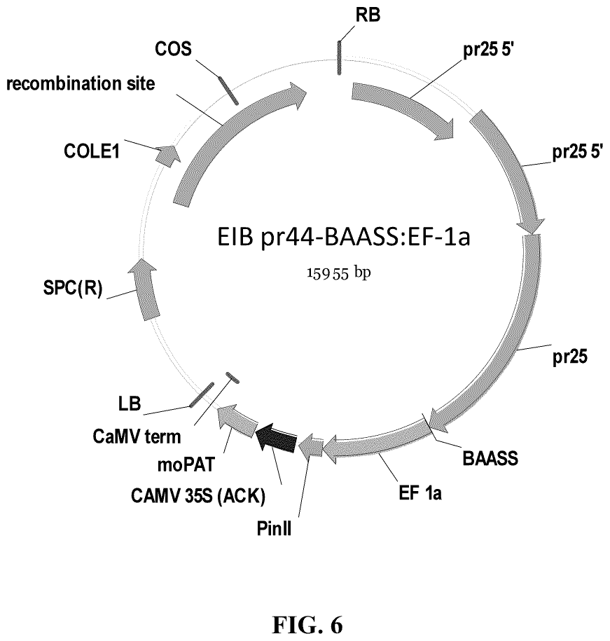

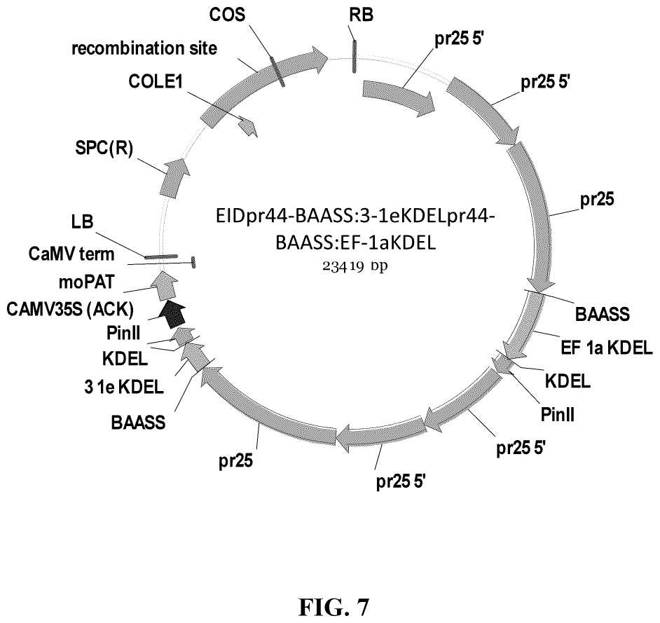

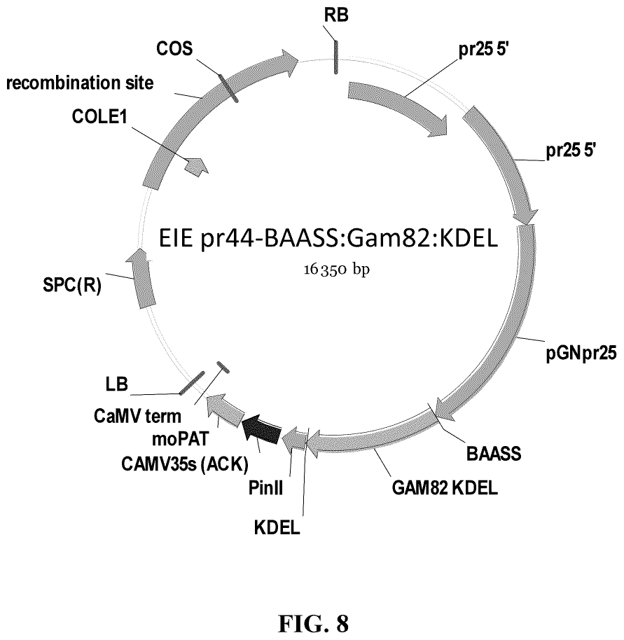

As shown in FIG. 1, constructs EIA and EIB were targeted to the apoplast/cell wall. EID and EIE were targeted to the endoplasmic reticulum (FIG. 1). As depicted in FIG. 1 and FIGS. 5-7, cellular/organelle-targeting sequences (BAASS:cell wall/apoplast) were included in all constructs. C-terminus sequences (KDEL:ER) were included in the codon-optimized EID and EIE constructs. The untranslated 3′ region of a potato protease inhibitor II (pinII) was also added to all constructs as a transcriptional termination element to enhance mRNA stability.

Embryo preferred promoter (Pr44:seed embryo) was used to drive high gene transcription and subsequent high yield of protein production in all constructs (also referred to herein as “expression cassettes”). The greatest protein expression levels were achieved using promoter pr44 in constructs containing single transcription units. Notably, promoter pr44 contains duplications of part of the well-characterized maize globulin-1 promoter.

### Transformation

After processing, the constructs were transferred into appropriate Agrobacterium strains for maize transformation. The constructs were transferred into Agrobacterium for maize transformation by a combination of triparental mating and electroporation as previously described¹³. In brief, a binary vector system for Agrobacterium was employed and then transferred to an Agrobacterium strain capable of transforming maize. Transformed cell lines were identified by herbicide resistance and allowed to mature into plants.

Specifically, Maize was transformed by first processing at least 2000 maize embryos per construct, followed by regeneration of TO generation plants. The plants were then transferred to a greenhouse and crossed with Hill untransformed maize to obtain T1 generation seed. The number of regenerated T1 generation plants for which seed was obtained for each construct is listed below. Notably, expression of individual transformation events in plants has been shown to vary widely among independent events; hence, we target many events per construct and many plants per event to mature and set seed.
EIA—89 plants representing 18 events
EIB—50 plants representing 4 events
EID—88 plants representing 15 events
EIE—37 plants representing 6 events

### Detection of Protein

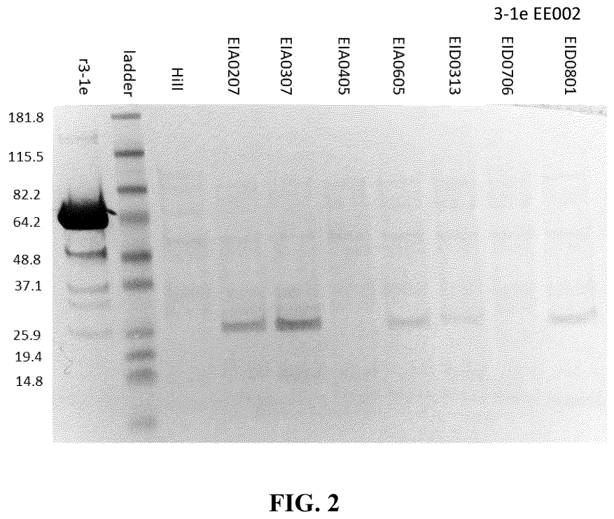

Large numbers of single seed in T1 generation plants were assayed for expression, yielding up to 360 samples per construct. Crude extracts were tested from ground whole seed. Optimal results were obtained with extraction in 1×PBS+0.05% tween. As shown by the representative Western Blots (FIGS. 2-4), robust expression of the three recombinant proteins (e.g., Eimeria vaccine proteins 3-1e, EF-1 alpha, and Gam82) was achieved. Western blots using anti-Eimeria protein antibodies were run on selected high expressing lines to confirm the size of the proteins and that they were immunoreactive with the Eimeria proteins.

3-1e

FIG. 2 shows a Western Blot of seed grain expression of 3-1e in constructs EIA and EID. Fifty whole seed were ground and extracted at 100 mg flour/1 mL 1× PBS+0.05% Tween. Each sample represents seed from one plant from an independent insertion event from either construct EIA or EID, with Hill as an untransformed negative control. Extract was loaded on a standard SDS PAGE gel under reducing conditions. The blot was transferred to membrane by iBlot and incubated overnight with mouse mAb to 3-1e at 1:1000. The blot was developed using anti-mouse IgG conjugated to alkaline phosphatase and BCIP/NBT. Expression was estimated at 100 mg/kg. Bands running larger than expected in Lane 1 are due to 3-1e aggregation and/or post-translational modifications.

### EF-1a

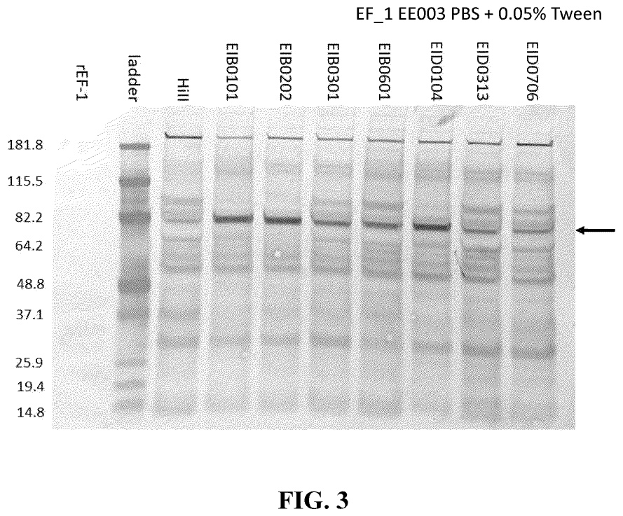

FIG. 3 shows Western Blot of seed grain expression of EF-1a in constructs EIB and EID. Fifty whole seed were ground and extracted at 100 mg flour/1 mL 1× PBS+0.05% Tween (pH 7.0). Each sample represents seed from one plant from an independent insertion event from either construct EIB or EID, with Hill as an untransformed negative control. Extract was loaded on a standard SDS PAGE gel under reducing conditions. The blot was transferred to membrane by iBlot and incubated overnight with rabbit Ab to EF-1a. The blot was developed using anti-rabbit conjugated to alkaline phosphatase and BCIP/NBT. Some degradation in transport was observed in the 1 μg of positive control recombinant protein utilized. The predicted size was 51 kD but the recombinant protein ran higher due to post-translational modifications (FIG. 3). The recombinant protein target appears at approximately 82 kD (indicated by an arrow in FIG. 3) due to said post-translational modifications. Expression is estimated to be an average of 50 mg/kg. Notably, the endogeneous maize EF-1a is about 70% identical to the Eimeria EF-1a, so there may be some cross-reactivity observed with the polyclonal antibody. Further analysis with single seeds is contemplated by the present inventors.

### Gam82

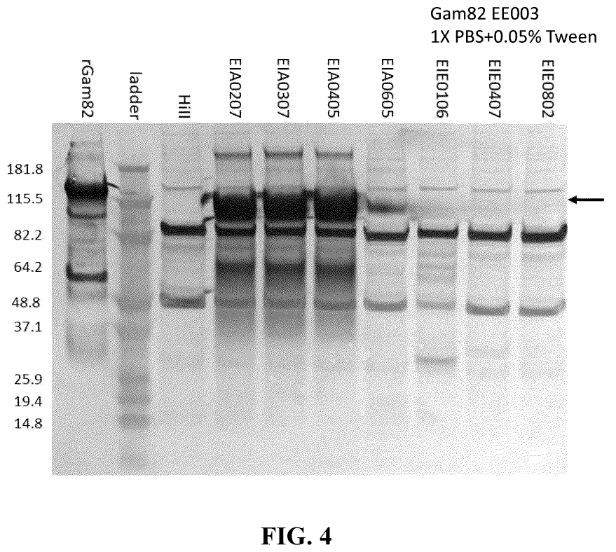

FIG. 4 is a Western Blot of seed grain expression of Gam82 in constructs EIA and EIE. Fifty whole seed were ground and extracted at 100 mg flour/1 mL 1× PBS+0.05% Tween. Each sample represents seed from one plant from an independent insertion event from either construct EIA or EIE, with Hill as an untransformed negative control. Extract was loaded on a standard SDS PAGE gel under reducing conditions. The blot was transferred to membrane by iBlot and incubated overnight with rAb to Gam82. The blot was developed using anti-rabbit conjugated to alkaline phosphatase and BCIP/NBT. There is a strong signal with the control E. coli recombinant protein and in several of the EIA lines (arrow), but they ran higher than expected due to post-translational modifications. The predicted size is about 67 kD. Based on this gel and comparison to the standard, expression is estimated up to 600 mg/kg. Further analysis with single seeds is contemplated by the present inventors.

Quantifying Expression with ELISA

Expression levels of the Eimeria vaccine proteins 3-1e polypeptide, Gam82 polypeptide, and/or EF-1 polypeptides in seed can also be quantitated by ELISA in a manner similar to that previously described¹⁶. In brief, individual seeds from each plant are extracted separately in PBS plus 1% Triton X-100 and the protein content in the extract measured by the Bradford method. The equivalent of 1 μg of extracted protein are then tested in an ELISA (Eimeria protein and anti-Eimeria protein antibodies may be derived by standard methods). Purified Eimeria protein is used as a standard and control seed extracts as a negative control. Six individual seeds per plant can be analyzed and the level of expression in positive seeds used to calculate the mean antigen expression level in each plant that in turn can be used to calculate the mean expression level of each event and finally used to calculate the mean expression of the various constructs. This approach can be used to select the constructs and the transformation events with the highest expression. This procedure has been statistically analyzed previously and shown to be sufficient for the selection of the highest expressing lines.

### Prepare Material for Further Testing

The present inventors selected lines with the highest expression level of Eimeria vaccine proteins 3-1e polypeptide, Gam82 polypeptide, and/or EF-1a polypeptides from the constructs. These constructs can be used for testing the Eimeria vaccine proteins individually. The seed can be propagated to select for high expressing stable lines with no unintended plant alterations. Material from subsequent generations can be used for the studies described herein. Said Eimeria vaccine proteins are expressed in high expressing stable lines at levels of at least 1 mg/kg of seed. Grain containing the Eimeria vaccine proteins can be used to purify the antigen using an antibody affinity column as described previously¹⁷. In brief, anti-Eimeria IgG may be attached to cyanogen bromide-activated Sepharose beads which can then be used to make a column. The column can be equilibrated with PBS and extracts loaded onto the column, washed with PBS and eluted with high salt at pH3. The eluate run on Western blots to confirm immunoreactivity and on Coomassie-stained gels to obtain an estimate of purity. In addition to Western blots, n-terminal sequence and lectin binding can be used to confirm glycosylation and spectrophotometric analysis can be used to compare the plant-produced protein with that of Eimeria proteins produced from other recombinant hosts. Grain from lines can be processed and used to form wafers as described previously¹⁸.

SEQUENCE DETAILS
(maize codon optimized sequences)
Bold: Initiating methionine
All caps. Stop codon
Underline: key restriction sites (NcoI, PacI, HindIII)
Italics: signal sequence (Barley alpha amylase (BAASS) and/or KDEL)
SEQ ID NO: 1
3-1e polypeptide sequence of GenBank AAD39362
(3-1e polypeptide is also referred to as “3-1e”, “3-1e protein” or “Eimeria acervulina 3-
1e/profilin protein sequence”)
BAASS 3-1e nucleotide sequence
SEQ ID NO: 2
accatggcgaacaagcacctgagccttagcctcttcctcgtgctcctgggcctctccgccagcctcgcctccggcggggaggaggcgg
acacccaggcctgggacacctcggtgaaggagtggctcgtcgacaccggcaaggtgtacgctggcggcatcgccagcatcgccgacg
gctgcaggctgttcggcgctgccatcgacaacggcgaggacgcctggtcccagctcgtcaagacgggctaccagatcgaggtgctgca
ggaggacggcagctccacgcaggaggactgcgacgaggctgagacgctgaggcaggccatcgtggacggcagggccccgaacggc
gtgtacatcggcggcatcaagtacaagcttgcggaggtcaagagggacttcacctacaacgaccagaactacgacgtggccatcctcgg
caagaacaagggcggcggcttcctcatcaagacgcccaacgacaacgtggtcatcgcgctctacgacgaggagaaggagcagaacaa
ggccgacgctctcacgactgctctcgctttcgcggagtacctctaccagggcggcttcTGATAGgttaacctagacttgtccatcttctg
gattggccaacttaattaatt
BAASS KDEL 3-1e nucleotide synthesis
SEQ ID NO: 3
Accatggcgaacaagcacctgagccttagcctcttcctcgtgctcctgggcctctccgccagcctcgcctccggcggggaggaggcg
gacacccaggcctgggacacctcggtgaaggagtggctcgtcgacaccggcaaggtgtacgctggcggcategccagcatcgccgac
ggctgcaggctgttcggcgctgccatcgacaacggcgaggacgcctggtcccagctcgtcaagacgggctaccagatcgaggtgctgc
aggaggacggcagctccacgcaggaggactgcgacgaggctgagacgctgaggcaggccatcgtggacggcagggccccgaacgg
cgtgtacatcggcggcatcaagtacaagcttgcggaggtcaagagggacttcacctacaacgaccagaactacgacgtggccatcctcg
gcaagaacaagggcggcggcttcctcatcaagacgcccaacgacaacgtggtcatcgcgctctacgacgaggagaaggagcagaaca
aggccgacgctctcacgactgctctcgctttcgcggagtacctctaccagggcggcttcaaggacgagctgTGATAGgttaacctag
acttgtccatcttctggattggccaacttaattaatt
SEQ ID NO: 4
EF-1a polypeptide sequence of GenBank XP_013234784
(EF-1a polypeptide is also referred to as “EF-1a”, “EF-1a protein” or “Eimeria tenella EF-
1a protein sequence”)
EF-1a nucleotide sequence
SEQ ID NO: 5
accatggcgaacaagcacctgagccttagcctcttcctcgtgctcctgggcctctccgccagcctcgcctccggcggcaaggagaaga
cgcacatcaacctggtggtcatcggccacgtggactccggcaagagcaccacgaccggccacctgatctacaagctcggcggcatcga
caagcgcaccatcgagaagttcgagaaggagtccagcgagatgggcaaggcgagcttcaagtacgcctgggtcctggacaagctcaag
gccgagagggagaggggcatcacgatcgacatcgcgctctggcagttcgagacgcccgccttccactacaccgtgatcgacgcccccg
gccacagggacttcatcaagaacatgatcaccggcaccagccaggggacgtcgccctcctggtggtgcccgcggaccagggcgggtt
cgagggcgcgttctccaaggaggggcagacgagggagcacgccctcctggccttcaccctgggcgtgaagcagatgatcgtcggcatc
aacaagatggacgccacgagccccgagaagtactccgaggcgcgcttcaacgagatccaggccgaggtgtcccgctacctcaagaccg
tcggctacaacccggagaaggtgccgttcgtccccatctccggcttcgtgggcgacaacatggtcgagcgctccagcaacatgggctggt
acaagggcaagaccctggtggaggccctggactcggtggagccgccaaagcgcccggtggacaagccactcaggctgccgctccaag
acgtctacaagatcggtggcatcggcaccgtgccggtgggcagggtggagacgggcgtcctgaagccgggcatggtggtcaccttcgc
cccctccggcctccagacggaggtgaagtccgtcgagatgcaccacgcccagctggagcaggcggtgcccggcgacaacgtgggctt
caacgtcaagaacgtgtccgtcaaggacgtgaagaggggccacgtcgcctccgacagcaagaacgacccggccaaggggccgcca
gctttcaggcccaggtcatcgtgctccaccaccccggccagatcaacccgggctacacccccgtgctggactgccacacggcccacatc
agctgcaagttcgcggacctggagaagaggctggacaggaggtcgggcaaggctctggaggactccccgaagagcatcaagtcgggc
gacgccgctatcgtgaggatggagccgagcaagcccatgtgcgtcgaggcgttcatcgagtacccgcccctcggcaggttcgccgtgag
ggacatgaagcagaccatcgcggtgggcgtcatcaaggccgtcgagaagaaggaggcgggcggcaaggtgacgaagtcggctcaga
aggccgcggctaagaagTGATAGgttaacctagacttgtccatcttctggattggccaacttaattaata
BAASS EF-1a KDEL nucleotide synthesis
SEQ ID NO: 6
accatggcgaacaagcacctgagccttagcctcttcctcgtgctcctgggcctctccgccagcctcgcctccggcggcaaggagaaga
cgcacatcaacctggtggtcatcggccacgtggactccggcaagagcaccacgaccggccacctgatctacaagctcggcggcatcga
caagcgcaccatcgagaagttcgagaaggagtccagcgagatgggcaaggcgagcttcaagtacgcctgggtcctggacaagctcaag
gccgagagggagaggggcatcacgatcgacategcgctctggcagttcgagacgcccgccttccactacaccgtgatcgacgcccccg
gccacagggacttcatcaagaacatgatcaccggcaccagccaggcggacgtcgccctcctggtggtgcccgcggaccaggggggtt
cgagggcgcgttctccaaggaggggcagacgagggagcacgccctcctggccttcaccctgggcgtgaagcagatgatcgtcggcatc
aacaagatggacgccacgagccccgagaagtactccgaggcgcgcttcaacgagatccaggccgaggtgtcccgctacctcaagaccg
tcggctacaacccggagaaggtgccgttcgtccccatctccggcttcgtgggcgacaacatggtcgagcgctccagcaacatgggctggt
acaagggcaagaccctggtggaggccctggactcggtggagccgccaaagcgcccggtggacaagccactcaggctgccgctccaag
acgtctacaagatcggtggcatcggcaccgtgccggtgggcagggtggagacgggcgtcctgaagccgggcatggtggtcaccttcgc
cccctccggcctccagacggaggtgaagtccgtcgagatgcaccacgcccagctggagcaggcggtgcccggcgacaacgtgggctt
caacgtcaagaacgtgtccgtcaaggacgtgaagaggggccacgtcgcctccgacagcaagaacgacccggccaaggcggccgcca
gctttcaggcccaggtcatcgtgctccaccaccccggccagatcaacccgggctacacccccgtgctggactgccacacggcccacatc
agctgcaagttcgcggacctggagaagaggctggacaggaggtcgggcaaggctctggaggactccccgaagagcatcaagtcgggc
gacgccgctatcgtgaggatggagccgagcaagcccatgtgcgtcgaggcgttcatcgagtacccgcccctcggcaggttcgccgtgag
ggacatgaagcagaccatcgcggtgggcgtcatcaaggccgtcgagaagaaggaggcgggcggcaaggtgacgaagtcggctcaga
aggccgcggctaagaaggacgagctgTGATAGgttaacctagacttgtccatcttctggattggccaacttaattaata
SEQ ID NO: 7
Gam82 polypeptide sequence of GenBank XP_013333566
(Gam82polypeptide is also referred to as “Gam82”, “Gam82 protein” or “Eimeria maxima Gam82
protein sequence”)
BAASS Gam82 nucleotide synthesis
SEQ ID NO: 8
accatggcgaacaagcacctgagccttagcctcttcctcgtgctcctgggcctctccgcctccctcgcctccggcgtcctgcctactgtgct
cgacacgaccacgggcacccaggtggagtggacggagacgccgctcgacaccacggaggtgactatgggcgagatgggctccacca
cgtctggcaccacgcccacctccacgggcgtccgcatgatggaggccgagacgacgaccccttccaccccggaggccccgcagcagc
agcagcagatgccccagccacagccccagccacagcagaccacgccggtgcccgaggcggtcctcgaggccatcatgcaggagatg
cagaacatcttcaggtccagcctggtgcccggctgggacaccgtgggcacagcggccgacgccgtgcgccagatcgtgacgcgcgtca
gggagaggctcacgggccccctgatgatgacggagatggacaccggtctcggcaggacgggccccctgtccaccaccggcgccaca
ggcgctaccaccggccccgtggccgcgctcaggggcgtcacgaacgacttcctgcgcgagatcatgatccaggaggcggtgctggag
acgctgtgggccgtggtcagggacgctcaggagcgcccgtggctggtgaacgagcaggaggccctgcacgctgccaccgccgacgc
cgtccagggcttccttggcaggatgcacgaccggctcagggctacgggcttcagcgaggaggaggtcatgaggctcctgccgaggtcc
cgcaacggcggctgcacgcgcaccggcggcctcttcgaccagtgcaacgacgccccgccaagcaggctcctgggcaagcgcatgtac
tccaccggctactacggctacggctacccctcctactacagctacggctactcctacccggcctacagccactaccccgtgtoctacccgta
ctacggctactcctggggcccgagctactactacggctctggctactacggcaagcacggctacaagtacggccactactaccgcaggct
cgccgagcaggagccgaggcccgtcatgccgccagccgctgcgaccgccgccgcgaacctgcgcgccgccgctgccgccgctgcc
gaggtgccgccaccgccaccgccagctgccgtcccgcccccaccgccagcagcggccgctggcaccccggcgatgatgccgccgcc
aatgatgggggtggaggagcccgtcccattccgcagcttgtacccctcctacagctggagctaccccgcttacacgcgcgtgagcccctc
ctacagctactacaccccgagctactccagctcctactactaccccaggtacaactacgcctacaactacccgctctactccgactacagct
ggtacgactacagctaccccctggcctacagctcctacagctcctacccgctctcctacagctcctactcctaccccctgagctacacctacc
cgtcggccttctaccgcaggctggaggtgcccgacctgaccacgaccacgaccacgcaccacgagcaacaacagcagcagcagcagg
agagcaccaccacggcggtgccaaccgagacgatcaccaccccgagcacgcgcaacacgcactccagctccctccgcaggggggc
gagcgctacgagcccatcacgccgacccagcgcaccttctacaacaacacggagggcaccaacaacccggtctacacgcccgagaac
ctgaccgaggatgagccgcagactgtgtgggagacctacaacTGATAGgttaacctagacttgtccatcttctggattggccaactta
attaatc
BAASS KDEL Gam82 nucleotide synthesis
SEQ ID NO: 9
accatggcgaacaagcacctgagccttagcctcttcctcgtgctcctgggcctctccgcctccctcgcctccggcgtcctgcctactgtgct
cgacacgaccacgggcacccaggtggagtggacggagacgccgctcgacaccacggaggtgactatgggcgagatgggctccacca
cgtctggcaccacgcccacctccacgggcgtccgcatgatggaggccgagacgacgaccccttccaccccggaggccccgcagcagc
agcagcagatgccccagccacagccccagccacagcagaccacgccggtgcccgaggcggtcctcgaggccatcatgcaggagatg
cagaacatcttcaggtccagcctggtgcccggctgggacaccgtgggcacagcggccgacgccgtgcgccagatcgtgacgcgcgtca
gggagaggctcacgggccccctgatgatgacggagatggacaccggtctcggcaggacgggccccctgtccaccaccggcgccaca
ggcgctaccaccggccccgtggccgcgctcaggggcgtcacgaacgacttcctgcgcgagatcatgatccaggaggcggtgctggag
acgctgtgggccgtggtcagggacgctcaggagcgcccgtggctggtgaacgagcaggaggccctgcacgctgccaccgccgacgc
cgtccagggcttccttggcaggatgcacgaccggctcagggctacgggcttcagcgaggaggaggtcatgaggctcctgccgaggtcc
cgcaacggcggctgcacgcgcaccggcggcctcttcgaccagtgcaacgacgccccgccaagcaggctcctgggcaagcgcatgtac
tccaccggctactacggctacggctacccctcctactacagctacggctactcctacccggcctacagccactaccccgtgtoctacccgta
ctacggctactcctggggcccgagctactactacggctctggctactacggcaagcacggctacaagtacggccactactaccgcaggct
cgccgagcaggagccgaggcccgtcatgccgccagccgctgcgaccgccgccgcgaacctgcgcgccgccgctgccgccgctgcc
gaggtgccgccaccgccaccgccagctgccgtcccgcccccaccgccagcagcggccgctggcaccccggcgatgatgccgccgcc
aatgatgggggtggaggagcccgtcccattccgcagcttgtacccctcctacagctggagctaccccgcttacacgcgcgtgagcccctc
ctacagctactacaccccgagctactccagctcctactactaccccaggtacaactacgcctacaactacccgctctactccgactacagct
ggtacgactacagctaccccctggcctacagctcctacagctcctacccgctctcctacagctcctactcctaccccctgagctacacctacc
cgtcggccttctaccgcaggctggaggtgcccgacctgaccacgaccacgaccacgcaccacgagcaacaacagcagcagcagcagg
agagcaccaccacggcggtgccaaccgagacgatcaccaccccgagcacgcgcaacacgcactccagctccctccgcaggggggc
gagcgctacgagcccatcacgccgacccagcgcaccttctacaacaacacggagggcaccaacaacccggtctacacgcccgagaac
ctgaccgaggatgagccgcagactgtgtgggagacctacaacaaggacgagctgTGATAGgttaacctagacttgtccatcttctg
gattggccaacttaattaatc

### Example 3

Obtain Protection from Challenge by Eimeria in Chickens

### Maize Grain Preparation for Feeding Trials

Seed from the lines with the highest levels of Eimeria vaccine proteins 3-1e, EF-1 alpha, and Gam82 were propagated to obtain grain for the feeding trials. The grain can be enriched for a germ fraction using a customized degerming system. In one embodiment, antigens were preferentially expressed in the embryo and enrichment can result in a 3-to-7-fold increase in antigen concentration. This approach has been used for many other proteins to increase recombinant protein concentrations. The germ fraction can be then ground into flour and defatted using SFE to increase stability. The germ flour can be integrated into a standard chicken diet using a targeted dose of 1 mg. The material can then be tested for stability of the antigens in dry feed at ambient temperatures. In alternate embodiments contemplated by the inventors, Eimeria vaccine proteins may be delivered individually or multiply to a subject animal. For example, two or more Eimeria vaccine proteins (e.g., 3-1e, EF-1 alpha, and Gam82) may be simultaneously delivered to a subject by mixing the various lines. The lines may also be crossed to provide additional means of delivering the target antigens.

### Evaluation of an Orally Delivered Eimeria Subunit Vaccine in Broiler Chickens:

Feeding and Challenge: Feeding and dosing can be arranged such that a dose sufficient to provide protection to a subject bird at the low end of consumption can be achieved. Anticipated dosing is such that herd immunity may be achieved with >85% of the birds protected. Based on animal models this should be sufficient to prevent economic loss and stop the spread of the disease.

The protective effect of corn-derived antigens of Eimeria delivered in a complete feed was evaluated in a vaccination-challenge study. A 3-day administration of the vaccine feed was administered at 7, 8 and 9 days of age and at 14, 15, and 16 days of age. There was also a non-vaccinated, non-challenge treatment group and a non-vaccinated, challenged treatment group. The details of the experimental design are summarized in Table 1 below.

TABLE 1
Experimental Design

| | | Eimeria | |
|---|---|---|---|
| group | Description | Challenge | Vaccine |
| 1 | Uninfected (negative) control | No | No |
| 2 | Positive control | Yes | No |
| 3 | High Dose | Yes | Yes - High |
| | | | Dose |
| 4 | Low Dose | Yes | Yes - Low |
| | | | Dose |

A large dose, mixed species Eimeria challenge consisting of approximately 35,000 oocysts of E. acervulina, E. maxima and E. tenella was administered at 28 days of age (study day 21). Lesion scoring was performed on day 6- and day 12 post-challenge. Treatments were compared for average lesion score (from 0 to 4, with score 2 lesions resulting in significant production losses), oocyst output, body weight and feed conversion using standard statistical analysis.

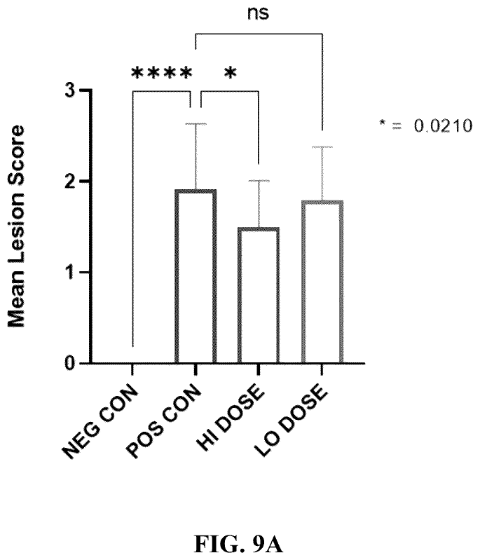

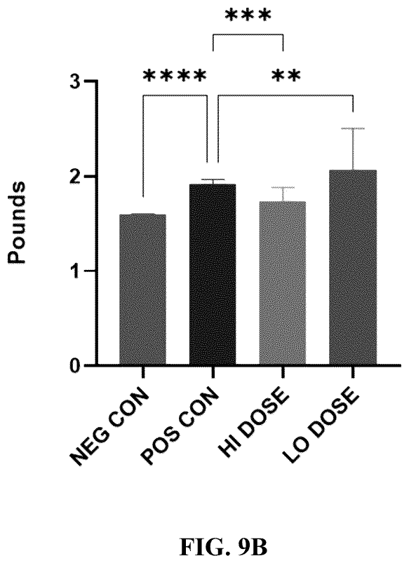

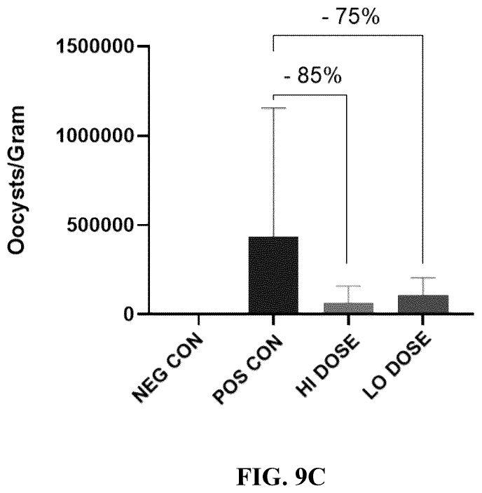

Results of the study showed a statistically significant protective response for the 3-day high dose vaccination regimen against E. acervulina challenge. Mean lesion score was reduced from 1.9 in the positive control group to 1.5 in the high dose group (FIG. 9A). When a categorical analysis of birds with lesion scores greater than or less than 2 was applied, there was a trend for the vaccinated groups to have fewer birds with lesions >2 as compared to the positive control for all species of Eimeria.

Performance data demonstrated that vaccination had no negative impact on body weight or feed conversion as compared to the negative control group. Vaccination provided no statistically significant protection against body weight loss or loss of feed conversion following challenge. However, weight gain post-challenge was better in the high dose vaccinated treatment group compared to the control groups (FIG. 9B).

Post-challenge oocyst output was also examined as a measure of immunity. Reduction in oocyst output reduces subsequent exposure to infection within a flock and reduces carry over of oocysts between flocks. The average number of oocysts per gram of excreta (OPG) was lower in both vaccine treatment groups at day 6 post-challenge as compared to the positive control, with an 85% and 75% reduction for the vaccinated group (FIG. 9C). These results are promising when compared to literature for subunit vaccines.

Overall, the orally delivered Eimeria subunit vaccine demonstrated safety and showed favorable results for immunogenicity. Future studies will be conducted to optimize antigen dose, timing of vaccine administration and to further characterize the immune response.

As described herein, the above example shows how an oral administration of maize-produced Eimeria antigens can protect chickens from challenge with Eimeria.

### Conclusions

Disclosed herein is a vaccine platform for the development of an orally delivered, low-cost, rapidly scalable Eimeria vaccine in chickens and other animals. To support this premise, the present inventors have demonstrated the following: 1) plants from forty-three independent transformation events were regenerated representing the three proteins 3-1e, EF-1 alpha, and Gam82 targeted to the apoplast or ER, 2). This is used to demonstrate that oral administration of maize-produced Eimeria antigens can protect chickens from a challenge against Eimeria with cost and scale-up advantages from production in plants. The initial development of an efficacious vaccine against avian coccidiosis may provide broad protection against several of the most prevalent Eimeria species. Western blot analysis was conducted on bulk seed from individual events. Levels of accumulation averaged 50 mg/kg whole seed for each of the antigens. This range is optimal to carry out animal studies without selection. Further, the present inventors contemplate expanding the highest producing lines such that the inventors can enrich for germ and bulk up grain. The inventors are now in a position to scale production to billions of doses in one year starting with only one ear of corn and at a fraction of the cost of traditionally produced vaccines. A heat-stable oral vaccine will also eliminate the need for the cold chain and personnel for administration enabling an easier and faster route towards mass immunization of poultry.

### Example 4

A study was conducted to demonstrate the feasibility of an oral vaccine candidate for chickens to provide protection against Eimeria spp. infection. Briefly, newly hatched broiler chickens were randomly housed in wire-floor battery cages with ad libitum access to feed and water throughout the study.

Zea mays grain containing vaccine from constructs EIA and EID was ground and assayed for antigen content. Vaccine material was stored between 2-25° C. Maize-expressed antigens against Eimeria spp. were delivered orally in-feed on d 0 to 3 and d 11 to 14 (T2 to T5). For a placebo control, commercial corn absent of the cocci antigens was grounded and also stored at 2-25° C. Live mixed Eimeria oocyst vaccine (i.e., ADVENT®, T6) and a combination of narasin ionophore and nicarbazin chemical (i.e., Maxiban®, T7) were provided to birds (domestic meat-type broiler chickens, Gallus gallus domesticus) according to manufacturer's instructions. On d 21, all birds were challenged with 12,500 sporulated oocysts of Eimeria maxima via oral gavage using 1 mL of challenge material per bird. On d 27, all birds were euthanized and dissected to quantify lesion score. Birds scored as 0 and 1 were considered negative for coccidiosis and scores 2, 3, and 4 were considered positive. In this study, vaccination with live mixed oocysts (T6) and or inclusion of narasin+nicarbazin (T7) failed to reduce lesion scores and provided Prevented Fraction (PF) not different from unvaccinated control (T1). Oral vaccination with Eimeria subunit vaccine expressed in maize (i.e., T2 to T5) reduced (P<0.001) coccidiosis lesion scores compared to unvaccinated control (T1). Furthermore, PF values for oral maize vaccination (T2 to T5) ranged from 0.872 to 0.989 with ranges for lower (LL) and upper (UL) 95% confidence intervals ranging from 0.778 to 0.937 for LL and 0.928 to 0.999 for UL. In conclusion, these results indicate that Eimeria subunit vaccine expressed in maize, when administered orally in feed, reduces the severity of coccidiosis infection in broiler chickens. Oral vaccination with antigen producing maize was highly effective at significantly reducing lesion scores. These results were confirmed by a very high and significant Prevented Fraction value.

Details of the study are given below.

### 1.0 Abbreviations

SD Study day.
ME Metabolizable energy.
CP Crude protein.
SID Standardized ileal digestible.
TSAA Total sulfur amino acids.
BW Body weight.
ADG Average daily gain.
ADFI Average daily feed intake.
FCR Feed conversion ratio.
GF Gain-to-feed efficiency.
EBI European broiler index.
MA Mortality adjusted.

### 2.0 Sequence of Events

TABLE 2

| | Study |
|---|---|
| Study Activity | Day |
| Daily facility/bird observations (recorded twice daily) | Daily |
| Recorded temperature and humidity | |
| Issued appropriate feed as necessary | |
| Weighed birds by pen | D 0 |
| Randomized birds to cages | |
| Weighed and issued non-vaccinated Starter feed to T1, T6, | |
| and T7 | |
| Weighed and issued Starter vaccinated feed to T2-T5 | |
| Sprayed coccidiosis vaccine to birds in T6 | |
| Weighed and issued non-vaccinated Starter feed to all cages | D 4 |
| Weighed back Starter feed to T2-T5 and discarded | D 11 |
| Issued vaccinated feed to T2-T5 | |
| Weighed birds by cage | D 14 |
| Weighed back Starter feed and reissued | |
| Issued Grower feed as needed | |
| Oral gavaged each bird with Eimeria maxima | D 21 |
| Weighed birds by cage | D 27 |
| Weighed back Grower feed and discard | |
| Coccidiosis lesion scored for all birds | |
| STUDY ENDED | |

### 3.0 Procedures

### 3.1. Treatments

TABLE 3

| | Maize | Live | | Treatment | | Birds | |
|---|---|---|---|---|---|---|---|
| | vaccine, | coccidiosis | | Duration, | No. of | per | Total |
| Code | mg/bird | vaccine* | Coccidiostat^{#} | days | cages | cage | Birds |
| T1 | 0 | No | No | 28 | 16 | 8 | 128 |
| T2 | 6.4 | No | No | 28 | 16 | 8 | 128 |
| T3 | 63.5 | No | No | 28 | 16 | 8 | 128 |
| T4 | 95.3 | No | No | 28 | 16 | 8 | 128 |
| T5 | 145.4 | No | No | 28 | 16 | 8 | 128 |
| T6 | 0 | Yes | No | 28 | 8 | 8 | 64 |
| T7 | 0 | No | Yes- | 28 | 8 | 8 | 64 |
| | | | Maxiban ® | | | | |
| Total | | | | | 96 | 8 | 768 |

*Mixed live oocyst vaccine ADVENT ® (Huvepharma, Inc., Peachtree City, GA) was used to spray vaccinate the chicks upon arrival to the animal room according to manufacturer's instructions.
^{#}A commercially available coccidiostat (i.e., Maxiban ®), was included in feed according to manufacturer's instructions from day of chick arrival to the animal room until the end of the study.

3.2. Experimental Design—The experimental design was a randomized block design of 12 cages per block for T2 to T5. A total of 4 blocks were used. Blocking was used to account for potential cage location variation within the animal room as well as the location of the cage within each battery unit. Treatment T1 was placed in a single battery unit to avoid any potential contamination of feed containing vaccine or ionophore that may spill from the feeders. Treatments T6 and T7 were housed in a single battery unit of 12 cages with T7 cages placed below T6 cages to avoid any potential contamination of feed containing ionophore that may spill from the feeders.

3.2.1. Animal Inclusion Criteria—Each bird met the following inclusion criteria: 1) Birds were clinically assessed to be in good health by the Study Investigator or designated personnel; 2) Coccidiosis vaccination from the hatchery did not occur.

3.2.2. Animal Exclusion Criteria—Animals not meeting the inclusion criteria outlined in Section 3.2.1 were excluded. Examples include pre-existing and existing conditions or disease (e.g., lameness, neurological disease, septicemia), unthrifty appearance, abnormal conformation, or history of numerous repeated antimicrobial treatments for disease or injury.

3.2.3. Animal Removal Criteria—No birds were removed from the trial for animal welfare reasons.

### 3.3. Experimental Unit—The Cage

### 3.4. Feeding Phases

TABLE 4

| Feeding | Study | | Feed | Feeding |
|---|---|---|---|---|
| Phase | Days | Treatments | Form | Program |
| Starter | 0 to 14 | T1 to T7 | Mash | Ad libitum |
| Grower | 14 to 27 | T1 to T7 | Mash | Ad libitum |
| Overall | 0 to 27 | T1 to T7 | Mash | Ad libitum |

3.4.1. Feed allowance for Starter feed containing vaccine corn were as follows:

TABLE 5

| | Starter Feed allowance, | Total feed to |
|---|---|---|
| Study days | g/bird for the 3-day period | be mixed, kg |
| 1, 2, and 3 | 51 | 26.1 |
| 11, 12, and 13 | 176 | 90.1 |

3.4.1.1. Feed Treatments were Provided as Follows, See Attachment #3 for More Details Regarding Treatment Mixing Sheets.

TABLE 6

| | Feed for Study | Feed for Study | Feed for Study |
|---|---|---|---|
| Code | Day 1, 2, and 3 | Day 4 to 10 | Day 11, 12, and 13 |
| T1 | Control Starter | Control Starter | Control Starter |
| T2 | AT2 | Control Starter | BT2 |
| T3 | AT3-T4-T5 | Control Starter | BT3 |
| T4 | AT3-T4-T5 | Control Starter | BT4 |
| T5 | AT3-T4-T5 | Control Starter | BT5 |
| T6 | Control Starter | Control Starter | Control Starter |
| T7 | Control Starter | Control Starter | Control Starter |

3.5. Randomization Procedures—Within each battery unit of 12 cages (i.e., a block), treatments T2 to T5 were randomly assigned. The randomization accounted for the vertical level of each cage and ensured that each treatment was represented at least once in each level of the battery unit. Similarly, treatments T6 and T7 were randomized within a single battery unit ensuring that each treatment was represented at least once in each level of the battery unit. All randomization procedures were performed using the rando function of Excel® (Microsoft Corporation, Redmond, WA).

3.6. Blinding Method—The study site personnel performing routine, husbandry, and experimental duties were blinded to treatments T2 to T7. The Study Investigator was not blinded to treatments. Treatments were be assigned a code for ease of administering treatment diets to the corresponding cages. Personnel evaluating lesion scores were blinded to all treatments.

### 3.7. Animal Management, Housing, and Equipment Qualification.

3.7.1. Facility Layout—Birds were housed within an environmentally controlled facility (Building 9) in Hazelton battery cages (17.5″ deep by 31″ wide by 13″ height) providing floor space & bird density of ˜0.471 ft²/bird (day 0). The animal facility has solid walls, solid concrete flooring, and mechanical ventilation. The facility has solid external access doors equipped with locks; doors are always locked. The facility has an anteroom to allow personnel to put disposable footwear, coveralls, and gloves as needed before entering the animal room, it also has trash cans to collect disposable wearables and a sink to wash hand before and after entering the animal room. Cages were arranged in sets of 12 cages; each battery set was equipped with 4 levels and each level had 4 cages. The bottom 4 cages of each unit were not used in this study. The separation between each battery unit was at least 2 feet. The room was equipped with vertical fluorescent devices to provide adequate light to all the levels of each battery unit.

3.7.2. Cage design and equipment—The size of each quadrangular stainless-steel cage was 39 inches wide by 26 inches long (i.e., 1,014 square inches of floor area) and had a wire floor. Each cage had one stainless steel self-feeder located in the front of the cage. Each cage had 3 low-pressure nipple waterers. The front of each cage had a card holder where the cage number was affixed. The card for each cage contained the treatment code. At the entrance of the room there was a card containing contact emergency information and study number.

3.7.3. Floor Space Allocation—At the end of the study (i.e., 27 days of age), each bird had a floor area allowance of 67.81 square inches.

3.7.4. Management and Environmental Conditions—Complied with Guide for the Care and Use of Agricultural Animals (4^{th} edition, FASS, 2020). Complied with any applicable institutional, local, state, and national regulations. Temperature and humidity were recorded daily and reported at the end of the study.

3.7.5. Lighting program—Lighting was 24-hour continuous light cycle via vertically mounted fluorescent lights for even illumination of all cages.

3.7.6. Temperature program—The facility was equipped with propane heaters to provide supplemental heat as needed throughout the animal room. These propane heaters were temperature controlled with temperature sensors and work synchronously with the ventilation program. Electric fans were also controlled by temperature sensors and automatically turned on when the programmed setpoint was reached to bring cooled outside air. The following table showed the targeted temperature for the animal room.

TABLE 7

| Bird age, days | Target Temperature, ° F. |
|---|---|
| 1 | 94 |
| 2 | 93 |
| 3 | 92 |
| 4 | 91 |
| 5 | 90 |
| 6 | 88 |
| 7 | 87 |
| 8 | 87 |
| 9 | 86 |
| 10 | 86 |
| 11 | 85 |
| 12 | 84 |
| 13 | 83 |
| 14 | 82 |
| 15 | 82 |
| 16 | 82 |
| 17 | 81 |
| 18 | 80 |
| 19 | 79 |
| 20 | 79 |
| 21 | 78 |
| 22 | 77 |
| 23 | 77 |
| 24 | 77 |
| 25 | 76 |
| 26 | 76 |
| 27 | 75 |
| 28 | 74 |

3.7.7. Ventilation program—Electrical fans were connected to timers and temperature-sensors. Fans were programed to run for a minimum of 60 seconds every 5 minutes to provide fresh outside air throughout the animal room or turned on when the temperature became out of range for birds at the respective age.

3.7.8. Scales—Scales used for weighing feed, feed additives, and birds were licensed by the State of Colorado. At each use, the scales were checked using standard weights according to CQR Standard Operating Procedures. This process was documented on a scale check weight form.

### 3.8. Feed and Water

3.8.1. Nutrient Requirements—Diets were formulated to meet or exceed the Nutrient Requirements of Poultry (1994) for meat-type chickens listed in Table 2-6 and took into consideration breeder's recommendations. Nutrient requirements used for diet formulation included, but are not limited to, ME, SID amino acids (i.e., lysine, threonine, and total sulfur), available phosphorus, calcium, sodium, chloride, trace minerals and vitamins.

3.8.2. Throughout the study animals had ad libitum access to feed via feed troughs unless otherwise indicated. Each cage had a separate feed trough, which was filled manually. Each cage had an individual feed bucket, labeled with the study number, treatment number, vaccinated or non-vaccinated (Starter phase only), and cage number. Feed for each cage was provided from the designated bucket as needed from day 0 to study end. Buckets were weighed back on days 14 and 27, weighed and recorded.

3.8.3. Water was provided ad libitum throughout the study via nipple waterers. Drinkers were checked twice daily to ensure a clean and constant water supply to the birds.

3.8.4. Diet Formulation—For each feeding phase, diets were formulated using a spreadsheet-based feed formulation using the nutrient recommendations obtained in Section 3.8.1. All nutrient and energy values for each ingredient used specific values for poultry.

TABLE 8

| Ingredients | Starter, % | Grower, % |
|---|---|---|
| Corn, yellow dent | 61.18 | 66.65 |
| Soybean meal, 47.5% CP | 33.95 | 28.50 |
| Soybean oil | 0.98 | 1.35 |
| L-lysine HCl | 0.24 | 0.23 |
| DL-methionine | 0.30 | 0.30 |
| L-threonine | 0.17 | 0.11 |
| Dicalcium phosphate 18.5% | 1.15 | 0.95 |
| Limestone | 1.15 | 1.07 |
| Salt | 0.25 | 0.25 |
| Sodium bicarbonate | 0.20 | 0.20 |
| Vitamin premix* | 0.15 | 0.15 |
| Trace mineral premix* | 0.16 | 0.15 |
| Choline Chloride 60% | 0.10 | 0.07 |
| Quantum Blue phytase | 0.02 | 0.02 |

*Provided to each kg of mixed diet: vitamin A 13,892 IU; vitamin D3 6,285 IU; vitamin E 74 IU; menadione 3.9 mg; riboflavin 13.1 mg; niacin 72.0 mg; pantothenic acid 19.5 mg; choline 600 mg; biotin 0.18 mg; vitamin B12 20.8 mg; folic acid 1.42 mg; pyridoxine 4.3 g; thiamine 3.1 mg; copper 14 mg; iodine 0.62 mg; iron 141 mg; manganese 106 mg; selenium 0.24 mg; and zinc 70 mg.

TABLE 9

| Nutrient | Starter | Grower |
|---|---|---|
| ME, kcal/kg | 3,000 | 3,081 |
| CP, % | 21.2 | 18.9 |
| Acid detergent fiber, % | 4.1 | 4.1 |
| Neutral detergent fiber, % | 8.6 | 8.6 |
| Crude fiber, % | 2.5 | 2.4 |
| Crude fat, % | 3.2 | 3.7 |
| SID Lys, % | 1.22 | 1.08 |
| SID Thr, % | 0.82 | 0.70 |
| SID Met, % | 0.62 | 0.59 |
| SID TSAA, % | 0.91 | 0.86 |
| SID Trp, % | 0.23 | 0.20 |
| SID Ile, % | 0.82 | 0.73 |
| SID Val, % | 1.06 | 0.95 |
| Ca, % | 0.90 | 0.82 |
| Available P, % | 0.45 | 0.41 |
| Na, % | 0.17 | 0.17 |
| Cl, % | 0.19 | 0.19 |
| Choline, mg | 2,241 | 1,966 |
| Ca:available P | 2.0 | 2.0 |
| SID Lys:ME | 4.06 | 3.49 |

3.8.5. Feed Manufacturing—For each feeding phase, a master batch was mixed at Colorado Quality Research from which all treatment diets (T1 to T7) were derived. Test article material was dynamically diluted with basal diet. Feed manufacturing records for all test feeds, as well as diet formulation, are included in this report.

3.8.6. Feed Manufacturing Records—All feed manufacturing batch records are included in this report.

3.8.7. Feed Labelling—The feed was stored in new feed sacks or buckets labeled with study number, feeding phase, vaccinated or non-vaccinated (Starter phase only) and treatment code.

### 3.9. Observations

3.9.1. Daily Observations—All animals were observed daily for changes in general health for the duration of the study. Daily observations were recorded.

### 3.10. Vaccination Procedure

3.10.1. Feed-vaccine doses are described in Section 3.1. Birds in T1, T6, and T7 received feed without vaccine throughout the course of the study. Birds in T2 to T5 were administered corn vaccine feed on days 0 to 3, and again on days 11 to 14 as outlined in Section 2.0. Vaccine feed was administered at a rate of 0.408 kg/cage (51 g/bird) for days 0 to 3, and 1.408 kg/cage (176 g/bird) for days 11 to 14. Vaccinated feed remained in all cages until the feed was completely consumed, at that point, birds received unvaccinated feed. On day 14, unconsumed feed was weighed for all treatments. Any unconsumed vaccinated Starter feed in T2 to T5 on day 14, was weighed and reissued until consumed and then Grower feed was issued as needed. On day 14, T1, T6, and T7 were issued Grower feed.

3.10.2. Spray Vaccine-Upon receipt (study day 0), birds in T6 only received live coccidiosis vaccine (1× dose) using a spray cabinet.

### 3.11. Challenge Procedure

3.11.1. The challenge material was prepared to deliver a target dose of 12,500 sporulated oocysts of Eimeria maxima via oral gavage using 1 mL of challenge material per bird. A single challenge pool was prepared to ensure all animals receive the same material.

### 3.12. Euthanasia, Necropsy and Lesion Scores

3.12.1. Euthanasia—Birds were euthanized using cervical dislocation as approved in the 2020 edition of the AVMA Guidelines for the Euthanasia of Animals.

3.12.2. Necropsy—Every bird found dead or euthanized during the study was weighed and necropsied by trained personnel. Findings were documented according to Attachment #1 and included in this report.

3.12.3. Lesion Scores—On day 27 of study (i.e., study termination), after cage weights, all birds from all cages were euthanized and dissected to determine Eimeria maxima lesion scores according to United States Department of Agriculture, Center for Veterinary Biologics, Veterinary Service Memorandum No. 800.123 citing the method of Johnson and Reid, 1970 (Experimental Parasitology, 28:30-36) and Diseases of Poultry 10^{th} edition, pages 878-883.

TABLE 10

| | | Coccidiosis |
|---|---|---|
| Eimeria maxima lesion scoring | | status |
| 0 | No gross lesions. | Negative |
| 1 | Small red petechiae may appear on the serosal side | Negative |
| | of the mid-intestine. There is no ballooning or | |
| | thickening of the intestine, though small amounts | |
| | of orange mucus may be present. | |
| 2 | Serosal surface may be speckled with numerous red | Positive |
| | petechiae; the intestine may be filled with orange | |
| | mucus; little or no ballooning of the intestine may | |
| | be present; and a thickening of the wall may be | |
| | observed. | |
| 3 | intestinal wall is ballooned and thickened. The | Positive |
| | mucosal surface is roughened; intestinal contents | |
| | are filled with pinpoint blood clots and mucus. | |
| 4 | The intestinal wall may be ballooned for most of | Positive |
| | its length; it contains numerous blood clots and | |
| | digested red blood cells are giving a characteristic | |
| | color and putrid odor; the wall is greatly thickened. | |
| | Dead animals are scored as 4. | |

### 3.13. Disposition of Vaccine, Feed, and Study Animals

3.13.1. Vaccine Corn—There was no unused vaccine material after preparing the experimental diets.

3.13.2. Feed—All unconsumed feed was composted on site observing all applicable local, state, and federal regulations and guidelines.

3.13.3. Study Animals—Disposal of all mortalities and birds euthanized during the study and at study end, were by dumpster/landfill. No birds from this study entered the human food supply or were rendered.

### 3.14. Outcome Variables

3.14.1. Primary Outcome—Eimeria maxima lesion scores as indicated in Section 3.12.3.

3.14.2. Secondary Outcome—Growth performance parameters.

TABLE 11

| List of Variables | Calculation for each cage |
|---|---|
| Average Starting Cage Live | Σ(Day 0 Cage weights)/(number of animals with a Day |
| Weight | 0 weight) |
| Average Live Final Weight (i.e., | Calculated using only initial and final weight data from |
| average weight on study Day 27 of | animals alive and completing Day 27: Σ(Cage animal |
| animals in a cage that were alive | weight Day 27 live weight)/(number of live animals |
| and completed Day 27) | with a Day 0 cage weight) |
| Average Final Body Weight (i.e., | [Σ(Cage weight Day 27 weights) + Σ(Cage animal |
| Day 27 weight or final withdrawal | Removal weights)]/(number of animals with a Day 0 |
| phase weight | cage weight) |
| Average body weight in | Cage weight of the Feeding Phase ÷ animal inventory |
| kilograms (Feeding Phase) | at the end of the Feeding Phase |
| Average body weight (Day 0) | Cage weight on day 0 ÷ animal inventory at day 0 |
| Average body weight (Day 14) | Cage weight on day 14 ÷ animal inventory at day 14 |
| Average body weight (Day 27) | Cage weight on day 27 ÷ animal inventory at day 27 |
| | (Average body weight at the end of the Feeding Phase − |
| Average Daily Gain, ADG in | average body weight at the start of the Feeding Phase) ÷ |
| grams (Feeding Phase) | number of days in the Feeding Phase) × 1,000 |
| ADG (Days 0 to 14) | (Average body weight at d 14 − average body weight at d |
| | 0) ÷ 14 days × 1,000 |
| ADG (Days 14 to 27) | (Average body weight at d 27 − average body weight at d |
| | 14) ÷ 14 days × 1,000 |
| ADG (Days 0 to 27) | (Average body weight at d 27 − average body weight at d |
| | 0) ÷ 27 days × 1,000 |
| Average Daily Feed Intake, | Total Feed Intake in the Feeding Phase ÷ Final animal |
| ADFI in grams (Feeding Phase) | inventory ÷ number of days in the Feeding Phase × 1,000 |
| ADFI (Day 0 to 14) | Total Feed Intake (Days 0-14) ÷ Final animal inventory |
| | (Day 14) ÷ 14 days × 1,000 |
| ADFI (Days 14 to 27) | Total Feed Intake (Days 14-27) ÷ Final animal inventory |
| | (Day 27) ÷ 14 days × 1,000 |
| ADFI (Days 0 to 27) | Total Feed Intake (Days 0-27) ÷ Final animal inventory |
| | (Day 27) ÷ 27 days × 1,000 |
| Unadjusted Feed conversion | ADFI of the Feeding Phase ÷ ADG of the Feeding |
| ratio, FCR (Feeding Phase) | Phase |
| FCR (Day 0 to 14) | ADFI (Day 0 to 14) ÷ ADG (Day 0 to 14) |
| FCR (Day 14 to 27) | ADFI (Day 14 to 27) ÷ ADG (Day 14 to 27) |
| FCR (Day 0 to 27) | ADFI (Day 0 to 27) ÷ ADG (Day 0 to 27) |
| | [(Average body weight at the end of the Feeding |
| | Phase ÷ age of animals in days at the end of the |
| European Broiler Index, EBI | Feeding Phase) × percent survival of the Feeding |
| (Feeding Phase) | Phase] ÷ (FCR of the feeding phase × 10) |
| EBI (Day 0 to 14) | [(Average body weight at d 14 ÷ 14 days) × percent |
| | survival at day 14] ÷ (FCR day 0 to 14 × 10) |
| EBI (Day 14 to 27) | [(Average body weight at d 27 ÷ 27 days) × percent |
| | survival at day 27] ÷ (FCR day 14 to 27 × 10) |
| EBI (Day 0 to 27) | [(Average body weight at d 27 ÷ 27 days) × percent |
| | survival at day 27] ÷ (FCR day 0 to 27 × 10) |
| | [(Cage weight at the end of the Feeding Phase − cage |
| | weight at the start of the Feeding Phase + Σ(Cage |
| Mortality Adjusted Average Daily | mortality weight)] ÷ [(animal inventory at the end of |
| Gain, in grams MA_ADG | the Feeding Phase + Σ(Animal days of mortality of the |
| (Feeding Phase) | Feeding Phase)] × 1,000 |
| MA_ADG (Days 0 to 14) | [(Cage weight at day 14 − cage weight at day 0 + Σ(Cage |
| | mortality weight days 0 to 14)] ÷ [(animal inventory at |
| | day 14 + Σ(Animal days of mortality of days 0 to 14)] × |
| | 1,000 |
| MA_ADG (Days 14 to 27) | [(Cage weight at day 27 − cage weight at day 14 + |
| | Σ(Cage mortality weight days 14 to 27)] ÷ [(animal |
| | inventory at day 27 + Σ(Animal days of mortality of |
| | days 14 to 27)] × 1,000 |
| MA_ADG (Days 0 to 27) | [(Cage weight at day 27 − cage weight at day 0 + Σ(Cage |
| | mortality weight days 0 to 27)] ÷ [(animal inventory at |
| | day 27 + Σ(Animal days of mortality of days 0 to 27)] × |
| | 1,000 |
| | Total Feed Intake in the Feeding Phase ÷ [(Final |
| Average Daily Feed Intake, | animal inventory × number of days in the Feeding |
| MA_ADFI in grams (Feeding | Phase) + Σ(Animal days of mortality of the Feeding |
| Phase) | Phase)] × 1,000 |
| MA_ADFI (Day 0 to 14) | Total Feed Intake days 0 to 14 ÷ [(Day 14 animal |
| | inventory × 14) + Σ(Animal days of mortality of days 0 |
| | to 14)] × 1,000 |
| MA_ADFI (Days 14 to 27) | Total Feed Intake days 14 to 27 ÷ [(Day 27 animal |
| | inventory × 14) + Σ(Animal days of mortality of days 14 |
| | to 27)] × 1,000 |
| MA_ADFI (Days 0 to 27) | Total Feed Intake days 0 to 27 ÷ [(Day 27 animal |
| | inventory × 27) + Σ(Animal days of mortality of days 0 |
| | to 27)] |
| Mortality Adjusted Feed | |
| conversion ratio, MA_FCR | MA_ADFI of the Feeding Phase ÷ MA_ADG of the |
| (Feeding Phase) | Feeding Phase |
| MA_FCR (Day 0 to 14) | MA_ADFI (Day 0 to 14) ÷ MA_ADG (Day 0 to 14) |
| MA_FCR (Day 14 to 27) | MA_ADFI (Day 14 to 27) ÷ MA_ADG (Day 14 to 27) |
| MA_FCR (Day 0 to 27) | MA_ADFI (Day 0 to 27) ÷ MA_ADG (Day 0 to 27) |

### 3.15. Conclusion Criterion

3.15.1. A reduction of lesion scores is interpreted to indicative effective vaccination.

### 3.15.2. Statistical Methods

Cage weights, feed issued, feed weigh back, and mortality data were entered into a spreadsheet. Growth performance and lesion score data were analyzed using the Fit Model function of JMP version 17.2 (JMP Statistical Discovery, LLC) with the following options: dependent variables (e.g., bird weights, lesion scores, etc.) as Role Variables, Model Effects containing treatment as the independent variable and block as a random effect, Personality as Standard Least Squares, Emphasis as Minimal Report, Method as REML. Multiple Least Square Means were compared using a two-tail t-test with Tukey HSD with α=0.05. Data were considered normally distributed if the Shapiro-Wilk test was greater than 0.05. No data transformations were performed.

As described in section 3.12.3, birds with lesion scores of 0 and 1 were considered negative for coccidiosis and assigned a value of 0 whereas lesion scores of 2, 3, and 4 were considered positive for coccidiosis and assigned a value of 1. Binomial coccidiosis rate (cocci rate) and mortality data were analyzed using the Generalized Linear Mixed Model add-in of JMP with the following options: Distribution as binomial, Link as logit, Fixed Effects as treatment, and Random Effects as block. Multiple Least Square Means were compared using a two-tail t-test with Tukey HSD with α=0.05. The transformed “logit” least square mean values (i.e, Xβ) were back transformed to obtain percent (i.e., p) cocci rate using:

$$p = \frac{exp ( X β )}{1 + exp ( X β )} \times 100$$

Cage served as the experimental unit for growth performance measurements, mortality, and lesion scores. Lesion score from all the birds within a cage will be averaged to obtain a cage lesion score value. Lesion scores and cocci rate were also analyzed using bird as the experimental unit. Prevented fraction (PF) values with lower (LL) and upper (UL) 95% confidence intervals were calculated separately calculated for each treated group (i.e., T2 to T7) against control (T1) in RStudio (2023.12.0+369) using the MN method of asymptotic intervals with RRsc( ), exact intervals with RRotsst( ), a logistic regression model with RRor( ) according to USDA CVB STATWI0007.01 using cage as the experimental unit.
>library (tidyverse)
>library (readxl)
>library (PF)
>setdw (“˜/folder_path”)
>Dat<—read_excel (“file_name.xlsx”)
>PF_mod<—RRor (glm(cocci/N˜factor(Trt)-1, family=quasibinomial, data=Dat))
>require (PF)
>RRsc(c(w,x,y,z))
>RRotsst(c(w,x,y,z))

Where: cocci is the number of coccidiosis positive birds in each cage.
Nis the total number of birds evaluated in each cage.
Trt is the treatment group code.
w is the number of coccidiosis positive birds in each treated group.
x is the total number of birds evaluated in each treated group.
y is the number of coccidiosis positive birds in control group (T1).
z is the total number of birds evaluated in control group (T1).

### 4.0 Adverse Event Recording

4.1. No abnormal conditions or behaviors were observed in this study.

### 5.0 Results

5.1. Lesion scores

TABLE 12

Average lesion score

| Code | By bird | By cage |
|---|---|---|
| T1 | 1.69^{a} | 1.69^{a} |
| T2 | 0.78^{b} | 0.80^{b} |
| T3 | 0.68^{b,c} | 0.68^{b} |
| T4 | 0.74^{b} | 0.74^{b} |
| T5 | 0.51^{c} | 0.51^{b} |
| T6 | 1.75^{a} | 1.74^{a} |
| T7 | 1.80^{a} | 1.80^{a} |
| SEM | 0.11 | 0.12 |
| P-value | <0.0001 | <0.0001 |

Average lesion score for each treatment group were calculated separately using pen or bird as the experimental unit. Least square means in the same column with different letter superscripts differ at P<0.05.

Results indicate that oral maize vaccine (i.e., T2 to T5) significantly reduced lesion scores compared to untreated control (T1). Vaccination with live mixed Eimeria oocyst vaccine (T6) or inclusion of a combination of a combination of narasin and nicarbazin in-feed (T7) failed to reduce lesion scores compared to control (T1).

5.2. Prevented fractions using logistic regression estimates, RRor( ).

TABLE 13

| | | | Cocci | Prevented Fraction | | |
|---|---|---|---|---|---|---|
| Code | N | Cocci | Positive Rate | PF | LL | UL |
| T1 | 126 | 91 | 0.722 | — | — | — |
| T2 | 119 | 11 | 0.092 | 0.859 | 0.583 | 0.952 |
| T3 | 121 | 1 | 0.008 | 0.989 | 0.830 | 0.999 |
| T4 | 117 | 8 | 0.068 | 0.931 | 0.728 | 0.982 |
| T5 | 124 | 5 | 0.040 | 0.943 | 0.802 | 0.984 |
| T6 | 56 | 43 | 0.768 | −0.039 | −0.397 | 0.228 |
| T7 | 64 | 52 | 0.813 | −0.122 | −0.463 | 0.139 |

PF results indicate that oral maize vaccine (i.e., T2 to T5) was highly significant at reducing Eimeria maxima coccidiosis in broiler birds. Vaccination with live mixed Eimeria oocyst vaccine (T6) or inclusion of a combination of a combination of narasin and nicarbazin in-feed (T7) did not ameliorate coccidiosis positive rate compared to control (T1).

5.3. Prevented fractions using asymptotic intervals using the MN method, RRsc( ).

TABLE 14

| | | | Prevented Fraction | | |
|---|---|---|---|---|---|
| Code | N | Cocci | PF | LL | UL |
| T1 | 126 | 91 | — | — | — |
| T2 | 119 | 11 | 0.872 | 0.778 | 0.928 |
| T3 | 121 | 1 | 0.989 | 0.937 | 0.998 |
| T4 | 117 | 8 | 0.905 | 0.819 | 0.952 |
| T5 | 124 | 5 | 0.944 | 0.873 | 0.976 |
| T6 | 56 | 43 | −0.063 | −0.261 | 0.132 |
| T7 | 64 | 52 | −0.125 | −0.316 | 0.056 |

PF results indicate that oral maize vaccine (i.e., T2 to T5) was highly significant at reducing Eimeria maxima coccidiosis in broiler birds. Vaccination with live mixed Eimeria oocyst vaccine (T6) or inclusion of a combination of a combination of narasin and nicarbazin in-feed (T7) did not ameliorate coccidiosis positive rate compared to control (T1).

5.4. Prevented fractions using exact intervals, RRtosst( ).

TABLE 15

| | | | Prevented Fraction | | |
|---|---|---|---|---|---|
| Code | N | Cocci | PF | LL | UL |
| T1 | 126 | 91 | — | — | — |
| T2 | 119 | 11 | 0.872 | 0.778 | 0.928 |
| T3 | 121 | 1 | 0.989 | 0.937 | −2.200 |
| T4 | 117 | 8 | 0.905 | 0.819 | 0.952 |
| T5 | 124 | 5 | 0.944 | 0.873 | 0.976 |
| T6 | 56 | 43 | −0.063 | −0.263 | 0.134 |
| T7 | 64 | 52 | −0.125 | −0.318 | 0.057 |

PF results indicate that oral maize vaccine (i.e., T2 to T5) was highly significant at reducing Eimeria maxima coccidiosis in broiler birds. Interestingly, the PF value for T3 from RRor( ), RRsc( ), and RRtosst( ) are all equal at 0.989 but the UL for T3 is negative at −2.200 with RRtosst( ) but highly positive for RRor( ) and RRsc( ) at 0.999 and 0.998, respectively. Vaccination with live mixed Eimeria oocyst vaccine (T6) or inclusion of a combination of a combination of narasin and nicarbazin in-feed (T7) did not ameliorate coccidiosis positive rate compared to control (T1).

5.5. Average body weight and average body weight gain.

TABLE 16

| | Average body weight, kg | | | Average daily gain, g | | |
|---|---|---|---|---|---|---|
| Code | D 0 | D 14 | D 27 | 0-14 | 14-27 | 0-27 |
| T1 | 0.0394 | 0.405 | 1.205 | 26.1 | 60.7 | 43.2 |
| T2 | 0.0396 | 0.383 | 1.173 | 24.5 | 60.7 | 42.0 |
| T3 | 0.0393 | 0.380 | 1.147 | 24.4 | 59.6 | 41.0 |
| T4 | 0.0392 | 0.387 | 1.196 | 24.8 | 62.2 | 42.8 |
| T5 | 0.0395 | 0.405 | 1.174 | 26.1 | 59.1 | 42.0 |
| T6 | 0.0388 | 0.398 | 1.203 | 25.6 | 62.2 | 43.1 |
| T7 | 0.0393 | 0.392 | 1.124 | 25.2 | 56.3 | 40.2 |
| SEM | 0.0002 | 0.010 | 0.023 | 0.7 | 1.3 | 0.9 |
| P-value | 0.2225 | 0.3125 | 0.2173 | 0.3078 | 0.1081 | 0.2162 |

Body weight gain and average daily weight gain were not different among treatments during Starter and Grower and the entire 27-d study.

### Attachment 1: Starter and Grower Feed Formulation

TABLE 17

| Ingredient | Starter, % | Grower, % |
|---|---|---|
| Corn | 61.18 | 66.65 |
| Soybean meal, 47.5% CP | 33.95 | 28.50 |
| Soybean oil | 0.98 | 1.35 |
| L-lysine HCl | 0.24 | 0.23 |
| DL-methionine | 0.30 | 0.30 |
| L-threonine | 0.17 | 0.11 |
| Dicalcium phosphate 18.5% | 1.15 | 0.95 |
| Limestone | 1.15 | 1.07 |
| Salt | 0.25 | 0.25 |
| Sodium bicarbonate | 0.20 | 0.20 |
| CQR vitamin premix | 0.15 | 0.15 |
| CQR trace mineral premix | 0.16 | 0.15 |
| Choline chloride 60% | 0.10 | 0.07 |
| Quantum Blue phytase | 0.02 | 0.02 |
| Total | 100.00 | 100.00 |

### Attachment 2: Treatment Mixing Sheets

### Dietary Treatments for Study Day 1, 2, and 3 (A)

Provide a total of 51 g of feed to each bird during this 3-day feeding period according to the table in Section 3.4.1.1.

TABLE 18

| | AT2 | | AT3-T4-T5 | |
|---|---|---|---|---|
| Ingredient | % | g | % | g |
| Vaccine corn-48.0 | 3.42 | 223.1 | 34.09 | 6,677.1 |
| Vaccine corn-35.2 | 2.31 | 151.0 | 23.09 | 4,521.0 |
| Corn, yellow dent | 55.45 | 3,619.6 | 4.00 | 783.3 |
| Soybean meal, 47.5% CP | 33.95 | 2,216.3 | 33.95 | 6,648.8 |
| Soybean oil | 0.98 | 64.0 | 0.98 | 191.9 |
| L-lysine HCl | 0.24 | 15.7 | 0.24 | 47.0 |
| DL-methionine | 0.30 | 19.6 | 0.30 | 58.8 |
| L-threonine | 0.17 | 11.1 | 0.17 | 33.3 |
| Dicalcium phosphate 18.5% | 1.15 | 75.1 | 1.15 | 225.2 |
| Limestone | 1.15 | 75.1 | 1.15 | 225.2 |
| Salt | 0.25 | 16.3 | 0.25 | 49.0 |
| Sodium bicarbonate | 0.20 | 13.1 | 0.20 | 39.2 |
| CQR vitamin premix | 0.15 | 9.8 | 0.15 | 29.4 |
| CQR trace mineral premix | 0.16 | 10.4 | 0.16 | 31.3 |
| Choline chloride 60% | 0.10 | 6.5 | 0.10 | 19.6 |
| Quantum Blue phytase | 0.02 | 1.3 | 0.02 | 3.9 |
| Total | 100.00 | 6,528.0 | 100.00 | 19,584.0 |

### Attachment 3: Treatment Mixing Sheets (Continued)

### Dietary Treatments for Study Day 11, 12, and 13 (B)

Provide a total of 176 g of feed to each bird during this 3-day feeding period according to the table in Section 3.4.1.1.

TABLE 19

| | BT2 | | BT3 | | BT4 | | BT5 | |
|---|---|---|---|---|---|---|---|---|
| Ingredient | % | g | % | g | % | g | % | g |
| Vaccine corn-48.0 | 0.99 | 223.0 | 9.88 | 2,225.8 | 19.76 | 4,451.7 | 35.36 | 7,965.7 |
| Vaccine corn-35.2 | 0.67 | 151.0 | 6.69 | 1,507.1 | 13.38 | 3,014.1 | 23.94 | 5,393.4 |
| Corn, yellow dent | 59.52 | 13,408.5 | 44.61 | 10,049.6 | 28.04 | 6,316.7 | 1.88 | 423.4 |
| Soybean meal, 47.5% CP | 33.95 | 7,648.3 | 33.95 | 7,648.3 | 33.95 | 7,648.3 | 33.95 | 7,648.3 |
| Soybean oil | 0.98 | 220.8 | 0.98 | 220.8 | 0.98 | 220.8 | 0.98 | 220.8 |
| L-lysine HCl | 0.24 | 54.1 | 0.24 | 54.1 | 0.24 | 54.1 | 0.24 | 54.1 |
| DL-methionine | 0.30 | 67.6 | 0.30 | 67.6 | 0.30 | 67.6 | 0.30 | 67.6 |
| L-threonine | 0.17 | 38.3 | 0.17 | 38.3 | 0.17 | 38.3 | 0.17 | 38.3 |
| Dicalcium phosphate 18.5% | 1.15 | 259.1 | 1.15 | 259.1 | 1.15 | 259.1 | 1.15 | 259.1 |
| Limestone | 1.15 | 259.1 | 1.15 | 259.1 | 1.15 | 259.1 | 1.15 | 259.1 |
| Salt | 0.25 | 56.3 | 0.25 | 56.3 | 0.25 | 56.3 | 0.25 | 56.3 |
| Sodium bicarbonate | 0.20 | 45.1 | 0.20 | 45.1 | 0.20 | 45.1 | 0.20 | 45.1 |
| CQR vitamin premix | 0.15 | 33.8 | 0.15 | 33.8 | 0.15 | 33.8 | 0.15 | 33.8 |
| CQR trace mineral premix | 0.16 | 36.0 | 0.16 | 36.0 | 0.16 | 36.0 | 0.16 | 36.0 |
| Choline chloride 60% | 0.10 | 22.5 | 0.10 | 22.5 | 0.10 | 22.5 | 0.10 | 22.5 |
| Quantum Blue phytase | 0.02 | 4.5 | 0.02 | 4.5 | 0.02 | 4.5 | 0.02 | 4.5 |
| Total | 100.00 | 22,528.0 | 100.00 | 22,528.0 | 100.00 | 22,528.0 | 100.00 | 22,528.0 |

### Attachment 4: Randomization

TABLE 20

Treatment

| Block | 2 | 3 | 4 | 5 |
|---|---|---|---|---|
| 1 | 33 | 36 | 34 | 35 |
| 2 | 39 | 40 | 37 | 38 |
| 3 | 43 | 44 | 42 | 41 |
| 4 | 47 | 46 | 48 | 45 |
| 5 | 52 | 50 | 49 | 51 |
| 6 | 53 | 54 | 56 | 55 |
| 7 | 58 | 57 | 60 | 59 |
| 8 | 64 | 63 | 62 | 61 |
| 9 | 66 | 68 | 67 | 65 |
| 10 | 72 | 71 | 69 | 70 |
| 11 | 74 | 76 | 75 | 73 |
| 12 | 80 | 79 | 77 | 78 |
| 13 | 82 | 84 | 81 | 83 |
| 14 | 85 | 88 | 87 | 86 |
| 15 | 90 | 89 | 92 | 91 |
| 16 | 93 | 95 | 94 | 96 |

TABLE 21

| | Treatment | | | | |
|---|---|---|---|---|---|
| Block | 1 | Block | 6 | Block | 7 |
| 17 | 1 | 18 | 17 | 19 | 21 |
| 17 | 2 | 18 | 18 | 19 | 22 |
| 17 | 3 | 18 | 19 | 19 | 23 |
| 17 | 4 | 18 | 20 | 19 | 24 |
| 17 | 5 | 18 | 25 | 19 | 29 |
| 17 | 6 | 18 | 26 | 19 | 30 |
| 17 | 7 | 18 | 27 | 19 | 31 |
| 17 | 8 | 18 | 28 | 19 | 32 |
| 17 | 9 | | | | |
| 17 | 10 | | | | |
| 17 | 11 | | | | |
| 17 | 12 | | | | |
| 17 | 13 | | | | |
| 17 | 14 | | | | |
| 17 | 15 | | | | |
| 17 | 16 | | | | |

### REFERENCES

[1] Dalloul R A, Lillehoj H S. Poultry coccidiosis: recent advancements in control measures and vaccine development. Expert Rev Vaccines. (2006).
[2] Antonissen G, Eeckhaut V, Van Driessche K, Onrust L, Haesebrouck F, Ducatelle R, et al. Microbial shifts associated with necrotic enteritis. Avian Pathol. (2016) 45:308-12.
[3] Chapman H D. Milestones in avian coccidiosis research: a review. Poult Sci. (2014) 93:501-11.
[4] Soutter, F., Werlin, D., Tomley, F., and Blake, D. Poultry Coccidiosis: Design and Interpretation of Vaccine Studies. Front. Vet. Sci., 26 Feb. (2020).
[5] Karavolias J, Salois M J, Baker K T, Watkins K. Raised without antibiotics: impact on animal welfare and implications for food policy. Transl Anim Sci. (2018) 2:337-48.
[6] Streatfield, S., Jilka, J., Hood, E., Turner, D., Bailey, M., Mayor, J., Woodard, S., Beifuss, K., Horn, M., and Delaney, D. (2001) Plant-based vaccines: unique advantages, Vaccine 19, 2742-2748.
[7] Holwerda, B. C., Padgett, H. S., and Rogers, J. C. (1992) Proaleurain vacuolar targeting is mediated by short contiguous peptide interactions, The Plant Cell 4, 307-318.
[8] Hayden, C. A., Egelkrout, E. M., Moscoso, A. M., Enrique, C., Keener, T. K., Jimenez-Flores, R., Wong, J. C., and Howard, J. A. (2012) Production of highly concentrated, heat-stable hepatitis B surface antigen in maize, Plant biotechnology journal 10, 979-984.
[9] Russell, D. A., and Fromm, M. E. (1997) Tissue-specific expression in transgenic maize of four endosperm promoters from maize and rice, Transgenic research 6, 157-168.
[10] An, G., Mitra, A., Choi, H. K., Costa, M. A., An, K., Thornburg, R. W., and Ryan, C. A. (1989) Functional analysis of the 3′ control region of the potato wound-inducible proteinase inhibitor II gene, The Plant Cell 1, 115-122.
[11] Tacket, C., Pasetti, M., Edelman, R., Howard, J., and Streatfield, S. (2004) Immunogenicity of recombinant LT-B delivered orally to humans in transgenic corn, Vaccine 22, 4385-4389.
[12] Lamphear, B., Barker, D., Brooks, C., Delaney, D., Lane, J., Beifuss, K., Love, R., Thompson, K., Mayor, J., and Clough, R. (2005) Expression of the sweet protein brazzein in maize for production of a new commercial sweetener, Plant biotechnology journal 3, 103-114.
[13] Streatfield, S. J., Bray, J., Love, R. T., Horn, M. E., Lane, J. R., Drees, C. F., Egelkrout, E. M., and Howard, J. A. (2010) Identification of maize embryo-preferred promoters suitable for high-level heterologous protein production, GM crops 1, 162-172.
[14] Xing, A., Moon, B. P., Mills, K. M., Falco, S. C., and Li, Z. (2010) Revealing frequent alternative polyadenylation and widespread low-level transcription read-through of novel plant transcription terminators, Plant biotechnology journal 8, 772-782.
[15] Hood, E., Bailey, M., Beifuss, K., Magallanes-Lundback, M., Horn, M., Callaway, E., Drees, C., Delaney, D., Clough, R., and Howard, J. (2003) Criteria for high-level expression of a fungal laccase gene in transgenic maize, Plant biotechnology journal 1, 129-140.
[16] Erlewyn-Lajeunesse, M., Brathwaite, N., Lucas, J., and Warner, J. (2009) Recommendations for the administration of influenza vaccine in children allergic to egg, Bmj 339, b3680.
[17] Shah, S., Hayden, C. A., Fischer, M. E., Rao, A. G., and Howard, J. A. (2015) Biochemical and biophysical characterization of maize-derived HBsAg for the development of an oral vaccine, Archives of biochemistry and biophysics 588, 41-49.
[18] Hayden, C. A., Smith, E. M., Turner, D. D., Keener, T. K., Wong, J. C., Walker, J. H., Tizard, I. R., Jimenez-Flores, R., and Howard, J. A. (2014) Supercritical fluid extraction provides an enhancement to the immune response for orally-delivered hepatitis B surface antigen, Vaccine 32, 1240-1246.
[19] Chabot, I., Goetghebeur, M. M., and Gregoire, J.-P. (2004) The societal value of universal childhood vaccination, Vaccine 22, 1992-2005.
[20] Hilleman, M. R. (2000) Vaccines in historic evolution and perspective: a narrative of vaccine discoveries, Vaccine 18, 1436-1447.
[21] Wilson, L. S., Reyes, C. M., Stolpman, M., Speckman, J., Allen, K., and Beney, J. (2002) The direct cost and incidence of systemic fungal infections, Value in Health 5, 26-34.
[22] Tarcha, E. J., Basrur, V., Hung, C.-Y., Gardner, M. J., and Cole, G. T. (2006) Multivalent recombinant protein vaccine against coccidioidomycosis, Infection and Immunity 74, 5802-5813.
[23] Smith, C. E., and Beard, R. R. (1946) Varieties of Coccidioidal Infection in Relation to the Epidemiology and Control of the Diseases, American Journal ofPublic Health and the Nations Health 36, 1394-1402.
[24] Nguyen, C., Barker, B. M., Hoover, S., Nix, D. E., Ampel, N. M., Frelinger, J. A., Orbach, M. J., and Galgiani, J. N. (2013) Recent advances in our understanding of the environmental, epidemiological, immunological, and clinical dimensions of coccidioidomycosis, Clinical Microbiology Reviews 26, 505-525.
[25] Pappagianis, D. (2001) Seeking a Vaccine against Coccidioides immitis and Serologic Studies: Expectations and Realities, Fungal Genetics andBiology 32, 1-9.
[26] Borchers, A. T., and Gershwin, M. E. (2010) The immune response in Coccidioidomycosis, Autoimmunity Reviews 10, 94-102.
[27] Fisher, B. T., Chiller, T. M., Prasad, P. A., Beveridge, M., Walsh, T. J., and Zaoutis, T. E. (2010) Hospitalizations for coccidioidomycosis at forty-one children's hospitals in the United States, The Pediatric Infectious Disease Journal 29, 243-247.
[28] CDC. (2013) Increase in reported coccidioidomycosis—United States, 1998-2011, Morbidity and Mortality Weekly Report 62, 217-221.
[29] Cole, G. T., Hung, C.-Y., Sanderson, S. D., Hurtgen, B. J., Wüthrich, M., Klein, B. S., Deepe, G. S., Ostroff, G. R., and Levitz, S. M. (2013) Novel strategies to enhance vaccine immunity against coccidioidomycosis, PLoS pathogens 9, e1003768.
[30] Delgado, N., Xue, J., Yu, J.-J., Hung, C.-Y., and Cole, G. T. (2003) A recombinant β-1,3-glucanosyltransferase homolog of Coccidioides posadasii protects mice against coccidioidomycosis, Infection and Immunity 71, 3010-3019.
[31] Herr, R. A., Hung, C.-Y., and Cole, G. T. (2007) Evaluation of two homologous proline-rich proteins of Coccidioides posadasii as candidate vaccines against coccidioidomycosis, Infect Immun 75, 5777-5787.
[32] Hurtgen, B. J., Hung, C.-Y., Ostroff, G. R., Levitz, S. M., and Cole, G. T. (2012) Construction and evaluation of a novel recombinant T cell epitope-based vaccine against coccidioidomycosis, InfectImmun 80, 3960-3974.
[33] Shubitz, L. F., Yu, J.-J., Hung, C.-Y., Kirkland, T. N., Peng, T., Perrill, R., Simons, J., Xue, J., Herr, R. A., and Cole, G. T. (2006) Improved protection of mice against lethal respiratory infection with Coccidioides posadasii using two recombinant antigens expressed as a single protein, Vaccine 24, 5904-5911.
[34] Howard, J. A., and Hood, E. E. (2007) Methods for Growing Nonfood Products in Transgenic Plants, Crop science 47, 1255-1262.
[35] Howard, J. A., and Hood, E. (2005) Bioindustrial and biopharmaceutical products produced in plants, In Advances in Agronomy (Sparks, D., Ed.), pp 91-124, Academic Press.
[36] Howard, J. A. (2005) Commercialization of biopharmaceutical and bioindustrial proteins from plants, Crop Science 45, 468-472.
[37] Kumar, G., Ganapathi, T., and Bapat, V. (2007) Production of hepatitis B surface antigen in recombinant plant systems: an update, Biotechnology progress 23, 532-539.
[38] Hayden, C. A. (2014) An oral vaccine for hepatitis B: challenges, setbacks, and breakthroughs, In Commercial Plant-Produced Recombinant Protein Products, pp 197-228, Springer.
[39] Streatfield, S. J. (2006) Mucosal immunization using recombinant plant-based oral vaccines, Methods 38, 150-157.
[40] Egelkrout, E., Rajan, V., and Howard, J. A. (2012) Overproduction of recombinant proteins in plants, Plant science 184, 83-101.
[41] Soria-Guerra, R. E., Moreno-Fierros, L., and Rosales-Mendoza, S. (2011) Two decades of plant-based candidate vaccines: a review of the chimeric protein approaches, Plant cell reports 30, 1367-1382.
[42] Hayden, C. A., Streatfield, S. J., Lamphear, B. J., Fake, G. M., Keener, T. K., Walker, J. H.,
Clements, J. D., Turner, D. D., Tizard, I. R., and Howard, J. A. (2012) Bioencapsulation of the hepatitis B surface antigen and its use as an effective oral immunogen, Vaccine 30, 2937-2942.
[43] Wüthrich, M., Gem, B., Hung, C. Y., Ersland, K., Rocco, N., Pick-Jacobs, J., Galles, K., Filutowicz, H., Warner, T., and Evans, M. (2011) Vaccine-induced protection against 3 systemic mycoses endemic to North America requires Th17 cells in mice, The Journal of clinical investigation 121, 554-568.
[44] Hung, C.-Y., Gonzalez, A., Wuthrich, M., Klein, B. S., and Cole, G. T. (2011) Vaccine immunity to coccidioidomycosis occurs by early activation of three signal pathways of T helper cell response (Th1, Th2, and Th17), Infect Immun 79, 4511-4522.
[45] Soto, E. R., Caras, A. C., Kut, L. C., Castle, M. K., and Ostroff, G. R. (2012) Glucan particles for macrophage targeted delivery of nanoparticles, Journal ofdrug delivery 2012.
[46] Tipper, D. J., and Szomolanyi-Tsuda, E. (2016) Scaffolded Antigens in Yeast Cell Particle Vaccines Provide Protection against Systemic Polyoma Virus Infection, Journal of immunology research 2016.
[47] Tacket, C. O., Pasetti, M. F., Edelman, R., Howard, J. A., and Streatfield, S. (2004) Immunogenicity of recombinant LT-B delivered orally to humans in transgenic corn, Vaccine 22, 4385-4389.
[48] Lamphear, B. J., Streatfield, S. J., Jilka, J. M., Brooks, C. A., Barker, D. K., Turner, D. D., Delaney, D. E., Garcia, M., Wiggins, B., and Woodard, S. L. (2002) Delivery of subunit vaccines in maize seed, Journal of Controlled Release 85, 169-180.
[49] Lamphear, B. J., Jilka, J. M., Kesl, L., Welter, M., Howard, J. A., and Streatfield, S. J. (2004) A corn-based delivery system for animal vaccines: an oral transmissible gastroenteritis virus vaccine boosts lactogenic immunity in swine, Vaccine 22, 2420-2424.
[50] Young, S. H., Ostroff, G. R., Zeidler-Erdely, P. C., Roberts, J. R., Antonini, J. M., and Castranova, V. (2007) A comparison of the pulmonary inflammatory potential of different components of yeast cell wall, Journal of toxicology and environmental health. Part A 70, 1116-1124.
[52] Lamphear, B. J., Streatfield, S. J., Jilka, J. M., Brooks, C. A., Barker, D. K., Turner, D. D., Delaney, D. E., Garcia, M., Wiggins, B., Woodard, S. L., Hood, E. E., Tizard, I. R., Lawhorn, B., and Howard, J. A. (2002) Delivery of subunit vaccines in maize seed, J Control Release 85, 169-180.
[53] Streatfield, S. J., Lane, J. R., Brooks, C. A., Barker, D. K., Poage, M. L., Mayor, J. M., Lamphear, B. J., Drees, C. F., Jilka, J. M., Hood, E. E., and Howard, J. A. (2003) Corn as a production system for human and animal vaccines, Vaccine 21, 812-815.

### SEQUENCE SUMMARY

SEQ ID NO: 1 3-1e polypeptide sequence of AAD39362
SEQ ID NO: 2 BAASS 3-1e nucleotide sequence
SEQ ID NO: 3 BAASS KDEL 3-1e nucleotide synthesis
SEQ ID NO: 4 EF-1a polypeptide sequence of GenBank XP_013234784
SEQ ID NO: 5 EF-1a nucleotide sequence
SEQ ID NO: 6 BAASS EF-1a KDEL nucleotide synthesis
SEQ ID NO: 7 Gam82 polypeptide sequence of GenBank XP_013333566
SEQ ID NO: 8 BAASS Gam82 nucleotide synthesis
SEQ ID NO: 9 BAASS KDEL Gam82 nucleotide synthesis

What is claimed is:

1. A method of producing a protective response to Eimeria in an animal, the method comprising:
a) orally administering to said animal a composition comprising plant or plant product comprising at least one Eimeria vaccine protein, or a sequence having at least 90% identity thereto or a functional fragment of said at least one Eimeria vaccine protein expressed at levels of at least 10 mg/kg in seed of said plant; and
b) producing a protective response to said Eimeria in said animal.

2. The method of claim 1, wherein said composition comprises plant material or plant tissue.

3. The method of claim 1, wherein said composition comprises seed or embryo of seed.

4. The method of claim 1, wherein said protective response comprises a serum antibody response in said animal.

5. The method of claim 4, wherein said serum antibody response is at least 4 times greater than serum antibody response in an animal not administered said vaccine.

6. The method of claim 1, wherein said at least one Eimeria vaccine protein comprises 3-1e polypeptide, Gam82 polypeptide, and/or EF-1a polypeptide.

7. The method of claim 6, wherein said at least one Eimeria vaccine protein is targeted to the apoplast or to the endoplasmic reticulum.

8. A vaccine comprising a plant-produced polypeptide of Eimeria vaccine protein, the vaccine comprising a plant or plant product comprising a construct comprising,
(a) a promoter preferentially directing expression to embryo tissue of a plant;
(b) a nucleic acid molecule encoding Eimeria vaccine protein as disclosed herein, or a sequence having at least 90% identity thereto or a functional fragment operably linked to said promoter; and
(c) a nucleic acid molecule targeting expression of said polypeptide in the apoplast or endoplasmic reticulum of said plant; and expressing said Eimeria vaccine protein in said plant at levels of at least 1 mg/kg of seed of said plant.

9. The vaccine of claim 8, wherein said construct comprises at least two Eimeria vaccine proteins, said Eimeria vaccine proteins comprising 3-1e polypeptide, Gam82 polypeptide, or EF-1a polypeptide.

10. The vaccine of claim 8, wherein said at least one Eimeria vaccine protein is not optimized for expression in said plant.

11. The vaccine of claim 8, wherein said construct comprises pr44-BAASS:3-1e pr44-BAASS:Gam82, pr44-BAASS:EF-1a, pr44-BAASS:3-1e KDEL pr44-BAASS:EF-1a KDEL, or pr44-BAASS:Gam82 KDEL.

12. The vaccine of claim 8, wherein Eimeria vaccine protein comprises 3-1e polypeptide, said 3-1e polypeptide comprising SEQ ID NO: 1 or is encoded by SEQ ID NO: 2 or 3.

13. The vaccine of claim 8, wherein Eimeria vaccine protein comprises EF-1a polypeptide, said EF-1a polypeptide comprising SEQ ID NO: 4 or is encoded by SEQ ID NO: 5 or 6.

14. The vaccine of claim 8, wherein Eimeria vaccine protein comprises Gam82 polypeptide, said Gam82 polypeptide comprising SEQ ID NO: 7 or is encoded by SEQ ID NO: 8 or 9.

15. A method of expressing a polypeptide of Eimeria vaccine protein or a functional fragment thereof, the method comprising introducing into a plant a construct comprising,
(a) a promoter preferentially directing expression to embryo tissue of a plant;
(b) a nucleic acid molecule encoding an Eimeria vaccine protein as disclosed herein or a sequence having at least 90% identity thereto or a functional fragment operably linked to said promoter; and
(c) a nucleic acid molecule targeting expression of said polypeptide in the apoplast and/or endoplasmic reticulum of said plant; and expressing said Eimeria vaccine protein in said plant at levels of at least 1 mg/kg of seed of said plant.

16. The method of claim 15, wherein said construct comprises construct EIA, construct EIB, construct EID, and/or construct EIE.

17. The method of claim 15, wherein said Eimeria vaccine protein comprises 3-1e polypeptide, said 3-1e polypeptide comprising SEQ ID NO: 1 or is encoded by SEQ ID NO: 2 or 3.

18. The method of claim 15, wherein said Eimeria vaccine protein comprises EF-1a polypeptide, said EF-1a polypeptide comprising SEQ ID NO: 4 or is encoded by SEQ ID NO: 5 or 6.

19. The method of claim 15, wherein said Eimeria vaccine protein comprises Gam82 polypeptide, said Gam82 polypeptide comprising SEQ ID NO: 7 or is encoded by SEQ ID NO: 8 or 9.

20. The method of claim 15, wherein said Eimeria vaccine protein is expressed in said plant or plant part at levels of at least 50 mg/kg.

21. The method of claim 15, wherein said Eimeria vaccine protein or fragment thereof is expressed in said plant or plant product at levels of at least 10 mg/kg of whole seed.
